SEQUENCE LISTING (10 sequences)

SEQ ID NO: 1 - DNA, 2131 bp, Homo sapiens
gtgagctcactcccgcctccatgttcccggagtcgcctggaagcgtccgcccaaggtcgcgggccgcttggggagtcagcagcgcgccaggccccttcgggccccacacgcattaggtgccttcttgatgggtacggagtgaacgcgggcggcggcgggaccgaggcagcgcccagtttgtaaccgccgcgccgcccgtgcccgcgcgcgccacaccccagcgcgcttccggccgggccacgtgaccgcgcgtgcacgtgttccggcctctccgcttcgccgctccgaacctcctcctggtcgtcccggcattcgtccacgcggagccggcttgggcggggcccgggaggcggcggccggagaagccgcggagacgcgagcgccgagcgtcgcgagggagcaggcccgggcaggcaagcggcggcctccgccatgaaccccaggggcctgttccaggacttcaaccccagtaagtttctcatctacacctgcctgctgctcttctcggtgctgctgcccctccgcctggacggcatcatccaatggagctactgggccgtctttgcccccatatggctgtggaagcttctagtcgtcgcaggcgcctccgtgggcgcgggcgtttgggcccgcaaccctcgctaccgcaccgagggagaggcctgtgtggagttcaaagccatgctgatcgctgtgggcatccacctgctgctgctcatgttcgaagtcctggtctgcgacagggtggagaggggcacccacttctggctgctggtcttcatgcctctcttcttcgtgtcccccgtgtccgtggctgcctgcgtctggggctttcgacacgataggtcgctggagctggagatcctgtgctcggtcaacatcctgcagttcatcttcatcgccctaaagctggacaggattattcactggccgtggctggtggtgtttgtgcccctgtggatcctcatgtcgttcctttgcctggtcgtcctctattacatcgtctggtccctcctgttcctgcggtccctggatgtggttgccgagcagcggagaacacacgtgaccatggctatcagttggataacgattgtcgtgcctctgctcacttttgaggtcctgctggttcacagattggatggccacaatacattctcctacgtctccatatttgtccccctttggctttccttactaactttaatggccacaacatttaggcgaaaggggggcaatcattggtggtttggcattcgcagagacttctgtcagtttctgcttgaaattttcccatttttaagagaatatgggaacatttcatatgatctccatcacgaagatagtgaagatgctgaagaaacatcagttccagaagctccgaaaattgctccaatatttggaaagaaggccagagtagttataacccagagccctgggaaatacgttcccccccctcccaagttaaatattgatatgccagattaaactcctagagaggacccaggcacacacagactccacttggccttcgcctcttgttcattcatcccaaacctggaaatggaaacaggcttcaaacactcgtctcacgccgtgtttgagatcaccgcctcatcagtatgcatcatagatggaggtggtttcagtatgtgggtgtgtgtgatgtgtacctgggtaagagacttgctttccaggttcgcactttcaggtgtagctgggggcagtaagtcgaattgttttagtaggtcctcaaaaggaataaccacacagctgtttgtttaaatgctactgtacctatcaaaactattgtttaaaaagtatttttatacactgctaatctaaaattgtatttcagattgtgcctgtcataacaatagcaaatgtaaaaagttctctttcccaccacttgtttataaacctcatagttgatatttttagtgttcctactgttaaaatactctctccttgggctttgctgatactggtctttaatattctgataggtgaatttttctaatggaatgaacccatgcatatatagtatttatatgaatattttagcagtgtaatatgttgaattctagttctctgcattaccattattacgttaaagtattttttaaagcttagg

SEQ ID NO: 2 - protein, 350 aa, Homo sapiens
MNPRGLFQDFNPSKFLIYTCLLLFSVLLPLRLDGIIQWSYWAVFAPIWLWKLLVVAGASVGAGVWARNPRYRTEGEACVEFKAMLIAVGIHLLLLMFEVLVCDRVERGTHFWLLVFMPLFFVSPVSVAACVWGFRHDRSLELEILCSVNILQFIFIALKLDRIIHWPWLVVFVPLWILMSFLCLVVLYYIVWSLLFLRSLDVVAEQRRTHVTMAISWITIVVPLLTFEVLLVHRLDGHNTFSYVSIFVPLWLSLLTLMATTFRRKGGNHWWFGIRRDFCQFLLEIFPFLREYGNISYDLHHEDSEDAEETSVPEAPKIAPIFGKKARVVITQSPGKYVPPPPKLNIDMPD

SEQ ID NO: 3 - protein, 350 aa, Mus musculus
MNPRGLFQDFNPSKFLIYACLLLFSVLLPLRLDGIIQWSYWAVFAPIWLWKLLVIVGASVGAGVWARNPRYRTEGEACVEFKAMLIAVGIHLLLLMFEILVCDRVERGTHFWLLVFMPLFFVSPVSVAACVWGFRHDRSLELEILCSVNILQFIFIALRLDRIIHWPWLVVFVPLWILMSFLCLVVLYYIVWSLLFLRSLDVVAEQRRTHVTMAISWITIVVPLLIFEVLLVHRLDDHNTFSYISIFIPLWLSLLTLMATTFRRKGGNHWWFGIRRDFCQFLLEVFPFLREYGNISYDLHHEDSEDAEDASVSEAPKIAPMFGKKARVVITQSPGKYVPPPPKLNIDMPD

SEQ ID NO: 4 - protein, 350 aa, Rattus norvegicus
MNPRGLFQDFNPSKFLIYACLLLFSVLLPLRLDGIIQWSYWAVFAPIWLWKLLVIVGASVGAGVWARNPRYRTEGEACVEFKAMLIAVGIHLLLLMFEILVCDRVERGTHFWLLVFMPLFFVSPVSVAACVWGFRHDRSLELEILCSVNILQFIFIALRLDRIIHWPWLVVFVPLWILMSFLCLVVLYYIVWSLLFLRSLDVVAEQRRTHVTMAISWITIVVPLLTFEVLLVHRLDGHNTFSYISIFTPLWLSLLTLMATTFRRKGGNHWWFGIRRDFCQFLLEVFPFLREYGNISYDLHHEDSEDAEDASALEAPKIAPMFGKKARVVITQSPGKYVPPPPKLNIDMPD

SEQ ID NO: 5 - protein, 351 aa, Danio rerio
MNLRGVFQDFNPSKFLIYACLLLFSVLLSLRLDGIIQWSYWAVFAPIWLWKLMVIIGASVGTGVWAHNPQYRAEGETCVEFKAMLIAVGIHLLLLTFEVLVCERVERASIPYWLLVFMPLFFVSPVSVAACVWGFRHDRSLELEILCSVNILQFIFIALKLDGIISWPWLVVCVPLWILMSFLCLVVLYYIVWSVLFLRSMDVIAEQRRTHITMAISWMTIVVPLLTFEILLVHKLDNHYSPNYVPVFVPLWVSLVTLMVTTFGQKGGNHWWFGIRKDFCQFLLELFPFLREYGNISYDLHHEDSDMSEELPIHEVPKIPTMFRKKTGVVITQSPGKYFVPPPKLCIDMPD

SEQ ID NO: 6 - protein, 358 aa, Drosophila melanogaster
MNLESLFRDFNPCKFIVHCSLFIFVTLFALRLDNYIDWPYWAIFTPLWIWKCTAILGAIVGAIVWCRYPHYRLEGDAYTQFKAMLISLALHLILLMFELLACDKLTSDRHLWVLVFIPLIFGSVVSVGACVWAVKHDRSFELELFLAVNALQFVSLPLKLDRFVYWNWDVVFVPMWIVICLSLVSVLYNIIFCGIMMRTPEVSLQQKKAALNAAVGNICTVLPLLCFQVVICDKLDGELKFPYIVVFSPLLVSILALIVLSSSAKGGNMWWFGIRKSFSQFLLSAMPFLQEYGNISYHPETHSNAAQSMPLDAASSSTSMAASEQLHEFHKHDKKSKKGAKNDILKPVVPFVSIDLPD

SEQ ID NO: 7 - DNA, 21 bp, Artificial
  siRNA strand comprising ribonucleic acids and deoxynucleic acids.
agcuggacaggauuauucatt

SEQ ID NO: 8 - DNA, 21 bp, Artificial
  siRNA strand comprising ribonucleic acids and deoxynucleic acids.
ugaauaauccuguccagcutt

SEQ ID NO: 9 - DNA, 21 bp, Artificial
  siRNA strand comprising ribonucleic acids and deoxynucleic acids.
uucuccuacgucuccauautt

SEQ ID NO: 10 - DNA, 21 bp, Artificial
  siRNA strand comprising ribonucleic acids and deoxynucleic acids.
auauggagacguaggagaatt

(19) United States
(12) Patent Application Publication
(10) Pub. No.: US 2005/0260642 A1
(43) Pub. Date: Nov. 24, 2005

(54) CNGH0011 polynucleotides, polypeptides, antibodies, and compositions, and methods of production and use

(76) Inventors: Chong Huang, Paoli, PA (US); Farhat Syed, Audubon, PA (US); Li Li, Downingtown, PA (US); Xilin Li, Wallingford, PA (US); Gregory Arndt, Malabar (AU); Susan Pond, Lindfield (AU)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/095,624

(22) Filed: Mar. 31, 2005

Related U.S. Application Data
(60) Provisional application No. 60/557,932, filed on Mar. 31, 2004.

(51) Int. Cl. C12Q 1/68; C07H 21/04; C12P 21/06; C12N 15/09; C07K 14/705; C07K 16/28

(52) U.S. Cl. 435/6; 435/69.1; 435/320.1; 435/325; 530/350; 530/388.22; 536/23.5

(57) ABSTRACT

CNGH0011 polypeptides and nucleic acids encoding a variety of proteins have diagnostic, preventive, therapeutic, and other uses for a number of human and other animal disorders. Nucleic acid antagonist molecules (siRNA, shRNA, among others), expression vectors containing nucleic acid molecules, host cells into which the expression vectors have been introduced, fusion polypeptides, antigenic peptides and antibodies can be utilized to control the level of protein and regulate a variety of cellular processes in diagnostic, screening, and therapeutic methods.

### CROSS-REFERENCE TO RELATED APPLICATION

This application claims the benefit of U.S. Provisional Application No. 60/557,932, filed Mar. 31, 2004, the entire disclosure of which is incorporated herein in its entirety.

### FIELD OF THE INVENTION

The present invention relates to CNGH0011 polypeptides, variants, or fragments thereof, and antibodies and anti-idiotype antibodies specific therefor, as well as nucleic acids encoding such CNGH0011 polypeptides, variants, fragments, antibodies, complementary nucleic acids, vectors, host cells, and methods of making and using thereof, including diagnostic and therapeutic formulations, administration and devices.

### BACKGROUND

With the recent completion of the sequencing and initial annotation of the human genome, and through the techniques of molecular biology, transgenic and null mutant or “knockout” animal production, computational biology, pharmacogenomics, and the like, the role and importance of individual genes and proteins in development and in normal and disease states is within the grasp of understanding.

With the tools enabling detection of the levels of gene expression and protein production in response to stimuli, natural or adverse, the practitioner is able to identify biological targets that are either representative of or critical to the change in physiological state of a cell, tissue or organism. From such knowledge, it is further possible to design molecules capable of identifying, quantitating, and modifying the levels of such target molecules. These newly designed molecules are the precursors of potential diagnostic, therapeutic, or protective agents.

Gene and protein sequence information relating to physiological processes enables the practitioner to assess, predict, and affect the physiological state of various human tissues.

Like many chronic obstructive pulmonary diseases (COPD's), asthma is a complex, chronic disorder, the presence and seriousness of which is determined by genetic and environmental factors. It is characterized by reversible airway obstruction, airway hyperresponsiveness, airway inflammation, and remodeling. Asthma impacts an estimated 15 million Americans. Its morbidity and mortality is increasing among industrialized countries.

Inflammation in the airway of an allergic asthmatic is associated with the mucosal infiltration of the T helper (Th) 2 subset of CD4+ T cells and eosinophils. The interaction between these cells leads to the production of various pro-inflammatory mediators involved in the pathogenesis of asthma.

Other forms of asthma are induced by exercise, viruses, aspirin, and occupational activities and environment. The mechanism for these forms of asthma may involve Th2 lymphocytes and cytokines as many chemokines and cytokines are known to be involved in the pathogenesis of asthma.

In particular, the Th2-derived cytokines, such as IL-4, IL-5, IL-9, and IL-13, play an important role in allergic diseases including asthma. However, these forms of asthma may be triggered differently than allergic asthma.

Microarray technology is a powerful tool for the simultaneous analysis of the expression of thousands of different genes. The process can be automated to enable a high-throughput format and output. Microarray technology is especially valuable in a multifactorial disease, such as asthma, because it can provide a gene expression profile. This is useful in the identification of novel genes, the function of genes of previously unknown function, and for the design of therapeutics and diagnostics.

The 7-transmembrane (7TM) receptor family is the fourth largest superfamily in the human genome with more than 800 known genes. Most of the family members activate heterotrimeric GTP binding proteins (G proteins), and belong to a superfamily of G-protein coupled receptors (GPCR's). Typically, the extracellular portions of the 7TM receptors bind a ligand and, in response to ligand binding, the cytosolic portions of these proteins activate a G-protein.

GPCR's consist of one single protein chain that crosses the membrane seven times, similar to the transmembrane seven-helix bundle of bacteriorhodopsin. Most ligands bind between the membrane helices, however, the periplasmic loops are sometimes also involved in ligand recognition. The second and third cytosolic loop and part of the (cytosolic) C-terminal end of the receptors are involved in G-protein recognition. GPCR's have diverse functions, detecting “inputs,” such as light, peptide hormones, neurotransmitters, the pheromones, odorants, morphogens, and chemoattractants, linking extracellular stimuli to intracellular signaling networks via heterotrimeric G proteins. Moreover, a growing body of evidence indicates that 7TM receptors can also transmit extracellular signals through mechanisms that function independently of G-protein coupling.

The nucleotide sequence of a gene labeled Family with sequence similarity 11, member B, Genbank Accession No. AF530474 (FAM11B) (SEQ ID NO: 1) and predicted amino acid sequence for which it codes (SEQ ID NO:2) were identified. The FAM11B nucleotide sequence was described as similar to the previously identified FAM11A gene. The FAM11B gene was hypothesized to be a transcribed retropseudogene from Chromosome 2.

Accordingly, there is a need to identify and characterize new 7TM polypeptides, polynucleotides, antibodies, or fragments that can be used to diagnose and treat COPD, such as asthma, emphysema, and bronchitis, and related diseases and conditions and overcome one more of these problems, as well as supplements to and improvements over known diagnostic and treatment methods and compositions.

### SUMMARY OF THE INVENTION

The present invention relates to isolated and/or recombinant CNGH0011 proteins and polypeptides and CNGH0011 polynucleotides. Preferred proteins and polypeptides possess at least one biological activity possessed by the corresponding naturally-occurring human polypeptide. Also within the invention are isolated CNGH0011 polypeptides or proteins having an amino acid sequence that is at least about 70%, preferably 75%, 80%, 90%, 95%, 99%, or 100% identical to SEQ ID NO: 2, and isolated polynucleotides encoding such polypeptides or proteins.

The present invention also relates to methods of treatment and/or diagnosis of chronic obstructive pulmonary disease (COPD), which for purposes of the present invention includes, without limitation, asthma, bronchitis, and emphysema, its symptoms, and conditions, as well as related diseases and conditions. The methods utilize (1) an isolated polypeptide comprising the sequence of SEQ ID NO: 2, variants, derivatives, and fragments thereof (collectively referred to herein as the “CNGH0011 polypeptide(s) or protein(s),” or “polypeptide(s) or protein(s) of the invention”), (2) an isolated polynucleotide comprising the sequence of SEQ ID NO: 1, variants, derivatives, and fragments thereof, and complementary sequences (collectively referred to herein as “CNGH0011 nucleic acids, polynucleotide(s), or gene(s),” or “nucleic acids, polynucleotide(s), or gene(s) of the invention”), and (3) an isolated polypeptide encoded by a polynucleotide comprising the sequence of SEQ ID NO: 1, variants and fragments thereof (also referred to herein as the “CNGH0011 polypeptide(s) or protein(s),” or “polypeptide(s) or protein(s) of the invention”) to diagnose and/or treat immune-mediated inflammatory diseases, such as COPD, their symptoms, and conditions, as well as related diseases and conditions. The CNGH0011 polynucleotides and polypeptides are useful as modulating agents in regulating a variety of cellular processes.

The present invention further provides nucleic acid molecules that resemble a double-stranded segment of the CNGH0011 gene sequence and its complement and can function in an inhibitory manner to the production of the polypeptides of the invention, such as siRNA molecules (SEQ ID NOS:7-10) or antisense molecules, short hairpin RNA (shRNA), other interfering RNA, or ribozymes.

The present invention also provides nucleic acid molecules that are suitable as primers or hybridization probes for the detection of nucleic acids encoding a polypeptide of the invention.

The invention includes nucleic acid molecules which encode naturally occurring allelic variants of a polypeptide having the amino acid sequence of SEQ ID NO: 2, wherein the nucleic acid molecule hybridizes under stringent conditions with a nucleic acid molecule having the nucleic acid sequence of either SEQ ID NO: 1 or a complement thereof.

Another aspect of the invention provides vectors, e.g., recombinant expression vectors, comprising a CNGH0011 polynucleotide. In another embodiment, the invention provides isolated host cells, e.g., mammalian and non-mammalian cells, containing such a vector and/or a CNGH0011 polynucleotide. The invention also provides methods for producing a CNGH0011 polypeptide by culturing, in a suitable medium, a host cell of the invention containing a recombinant expression vector encoding a CNGH0011 polypeptide such that the polypeptide is produced.

In one embodiment, a polypeptide of the invention has an amino acid sequence sufficiently identical to an identified domain of a CNGH0011 polypeptide. As used herein, the term “sufficiently identical” refers to a first amino acid or nucleotide sequence which contains a sufficient or minimum number of identical or equivalent (e.g., with a similar side chain) amino acid residues or nucleotides to a second amino acid or nucleotide sequence such that the first and second amino acid or nucleotide sequences have a common domain and/or common functional activity.

In one embodiment, the isolated CNGH0011 polypeptide represents a receptor protein containing an extracellular domain, a transmembrane domain, and an intracellular domain. In another embodiment, the CNGH0011 polypeptide contains a truncated or domain deleted extracellular portion. In another embodiment, the CNGH0011 polypeptide lacks a cytoplasmic domain or a portion thereof. In another embodiment, the polypeptide lacks both a transmembrane domain and a cytoplasmic domain and is soluble under physiological conditions.

The nucleic acids coding for the CNGH0011 polypeptides, or fragments thereof, can be operably linked to a nucleic acid for a heterologous amino acid sequence to form a fusion protein. A further embodiment is a “mimetibody” which comprises at least a fragment of a CNGH0011 polypeptide fused to at least one immunoglobulin constant region including a mimetibody wherein the immunoglobulin constant region is the Fc portion of human IgG1.

In another aspect, the present invention relates to antibodies and antibody fragments capable of binding to (a) a polypeptide having at least 70% amino acid sequence identity to all or part of the amino acid sequence of SEQ ID NO: 2 or (b) a polypeptide encoded by a polynucleotide having at least 70% nucleic acid identity to all or part of the polynucleotide sequence of SEQ ID NO: 1. In addition, the invention comprises antibody compositions, formulations, devices, transgenic mice and plants. The present invention also provides methods for generating and characterizing human, primate, rodent, mammalian, chimeric, single chain, humanized and/or CDR-grafted anti-CNGH0011 antibodies, immunoglobulins, cleavage products and other specified portions and variants thereof.

The present invention further provides at least one CNGH0011 anti-idiotype antibody to at least one CNGH0011 antibody of the present invention.

The present invention also provides at least one method for expressing a CNGH0011 peptide, anti-CNGH0011 antibody, or CNGH0011 anti-idiotype antibody, in a host cell, comprising culturing a host cell as described herein under conditions wherein at least one such CNGH0011 peptide, anti-CNGH0011 antibody, or CNGH0011 anti-idiotype antibody is expressed.

In yet another aspect, the present invention provides a fusion protein comprising a peptide which has at least 70% amino acid sequence identity to the amino acid sequence of SEQ ID NO: 2, fused to a heterologous amino acid sequence.

In another aspect, the present invention provides methods for detecting the presence of the activity or expression of a CNGH0011 polypeptide in a biological sample by contacting the biological sample with an agent or agents capable of detecting the presence or the activity of CNGH0011 in the biological sample.

In another aspect, the invention provides methods for modulating activity of a CNGH0011 polypeptide comprising contacting (or administering to) a cell with an agent (e.g., antagonist or agonist) that modulates (inhibits or enhances) the activity or expression of a CNGH0011 polypeptide such that activity or expression in the cell is modulated. In a preferred embodiment, the agent is an antibody that specifically binds to a polypeptide of the invention. In other embodiments, the modulator is a peptide, peptidomimetic, or other small molecule.

The present invention also provides methods of treating a subject having a disorder wherein the disorder can be ameliorated by modulating the amount or activity of the CNGH0011 polypeptide. The present invention also provides methods of treating a subject having a disorder characterized by increased or decreased activity of a CNGH0011 polypeptide or increased or decreased expression of a CNGH0011 polynucleotide by administering to the subject an agent that is a modulator of the activity of a CNGH0011 polypeptide or a modulator of the expression of a CNGH0011 polynucleotide (e.g., an antagonist or agonist). In one embodiment, the modulator is a CNGH0011 polypeptide. In another embodiment, the modulator is a CNGH0011 polynucleotide.

The present invention also provides diagnostic assays for identifying the presence or absence of a genetic lesion or mutation including, without limitation: (i) aberrant modification or mutation of a gene encoding a CNGH0011 polypeptide, (ii) mis-regulation of a gene encoding a CNGH0011 polypeptide, and (iii) aberrant post-translational modification of a CNGH0011 polypeptide wherein a wild-type form of the gene encodes a polypeptide having the activity of the CNGH0011 polypeptide.

In another aspect, the invention provides a method for identifying a compound that binds to or modulates the activity of a CNGH0011 polypeptide. In general, such methods entail measuring a biological activity of the polypeptide in the presence and absence of a test compound and identifying those compounds that alter the activity of the polypeptide.

The invention also features methods for identifying a compound that modulates expression of a CNGH0011 polypeptide or polynucleotide by measuring expression of the polypeptide or nucleic acid in the presence and absence of the compound.

The present invention also provides at least one composition comprising (a) an isolated CNGH0011 polypeptide, polynucleotide, and/or antibody as described herein; and (b) a suitable carrier or diluent. The carrier or diluent can optionally be pharmaceutically acceptable, according to known carriers or diluents. The composition can optionally further comprise at least one further compound, protein, or composition.

The present invention further provides any invention described herein.

### DESCRIPTION OF THE FIGURES

FIG. 1 shows the predicted domains of a CNGH0011 polypeptide.

FIG. 2 shows a multiple sequence alignment of the CNGH0011, CNGM0011, CNGR0011, FAM11A, CNGZ0011, and CNGD0011 polypeptides.

FIG. 3 shows a phylogenetic tree for the CNGH0011, CNGM0011, CNGR0011, FAM11A, CNGZ0011, and CNGD0011 polypeptides.

### DESCRIPTION OF THE INVENTION

The following definitions are set forth to illustrate and define the meaning and scope of various terms used to describe the invention herein.

An “activity,” a biological activity, and a functional activity of a polypeptide refer to an activity exerted by a CNGH0011 protein or polypeptide in response to its specific interaction with another protein or molecule as determined in vivo, in situ, or in vitro, according to standard techniques. Such activities can be a direct activity, such as an association with or an enzymatic activity on a second protein, or an indirect activity, such as a cellular process mediated by interaction of the protein with a second protein or a series of interactions as in intracellular signalling or the coagulation cascade.

An “antibody” includes any polypeptide or peptide containing molecule that comprises at least a portion of an immunoglobulin molecule, such as but not limited to, at least one complementarity determining region (CDR) of a heavy or light chain or a ligand binding portion thereof, a heavy chain or light chain variable region, a heavy chain or light chain constant region, a framework region, or any portion, fragment or variant thereof. The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. For example, antibody fragments include, but are not limited to, Fab (e.g., by papain digestion), Fab′ (e.g., by pepsin digestion and partial reduction) and F(ab′)2 (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention (see, e.g., Colligan, et al., eds., Current Protocols in Immunology, John Wiley & Sons, Inc., NY (1994-2001); Colligan et al., Current Protocols in Polypeptide Science, John Wiley & Sons, NY (1997-2001)).

“Chimeric” or “fusion” molecules are nucleic acids or polypeptides that are created by combining one or more of CNGH0011 polynucleotides (or their parts) with additional nucleic acid sequence(s). Such combined sequences may be introduced into an appropriate vector and expressed to give rise to a chimeric or fusion polypeptide.

“Complement of” or “complementary to” a nucleic acid sequence of the invention refers to a polynucleotide molecule having a complementary base sequence and reverse orientation as compared to a first polynucleotide.

“Fragment” is a variant polypeptide having an amino acid sequence that is entirely the same as part but not all of any amino acid sequence of a CNGH0011 polypeptide or a variant polynucleotide having a nucleic acid sequence that is entirely the same as part but not all of any nucleic acid sequence of any CNGH0011 polynucleotide. Fragments can include, e.g., truncation polypeptides having a portion of an amino acid sequence as shown in the amino acid sequence of SEQ ID NO: 2, or of variants thereof, such as a continuous series of residues that includes a heterologous amino- and/or carboxy-terminal amino acid sequence. Degradation forms of the CNGH0011 polypeptides produced by or in a host cell are also included. Other exemplary fragments are characterized by structural or functional attributes such as fragments that comprise alpha-helix or alpha-helix forming regions, beta-sheet or beta-sheet forming regions, turn or turn-forming regions, coil or coil-forming regions, hydrophilic regions, hydrophobic regions, alpha-amphipathic regions, beta-amphipathic regions, flexible regions, surface-forming regions, substrate binding regions, extracellular regions, and high antigenic index regions.

Further exemplary fragments include an isolated polypeptide comprising an amino acid sequence having at least 15, 20, 30, 40, 50 or 100 contiguous amino acids from the amino acid sequence set forth in SEQ ID NO: 2, or an isolated polypeptide comprising an amino acid sequence having at least 15, 20, 30, 40, 50 or 100 contiguous amino acids truncated or deleted from the amino acid sequence set forth in SEQ ID NO: 2. Fragments also include isolated polynucleotides having similar sizes and characteristics.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing:Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). In addition, values for percentage identity can be obtained from amino acid and nucleotide sequence alignments generated using the default settings for the AlignX component of Vector NTI Suite 8.0 (Informax, Frederick, Md.).

Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990)). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity.

Preferred parameters for polypeptide sequence comparison include the following:
(1) Algorithm: Needleman and Wunsch, J. Mol. Biol. 48:443-453 (1970) Comparison matrix:
BLOSSUM62 from Hentikoff and Hentikoff, Proc. Natl. Acad. Sci, USA. 89:10915-10919 (1992)
Gap Penalty: 12
Gap Length Penalty: 4
   A program useful with these parameters is publicly available as the “gap” program from Genetics Computer Group, Madison Wis. The aforementioned parameters are the default parameters for peptide sequence comparisons (along with no penalty for end gaps).

Preferred parameters for polynucleotide comparison include the following:
(1) Algorithm: Needleman and Wunsch, J. Mol. Biol. 48:443-453 (1970)
Comparison matrix: matches=+10, mismatch=0
Gap Penalty: 50
Gap Length Penalty: 3
   Available as: The “gap” program from Genetics Computer Group, Madison Wis. These are the default parameters for nucleic acid sequence comparisons.

By way of example, a polynucleotide sequence may be identical to the sequence of SEQ ID NO: 1, that is be 100% identical, or it may include up to a certain integer number of nucleotide alterations as compared to the reference sequence. Such alterations are selected from the group consisting of at least one nucleotide deletion, substitution, including transition and transversion, or insertion, and wherein the alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in SEQ ID NO: 1 by the numerical percent of the respective percent identity (divided by 100) and subtracting that product from the total number of nucleotides in SEQ ID NO: 1, or:
n.sub.n.ltorsim.x.sub.n-(x.sub.n.y),
   wherein n.sub.n is the number of nucleotide alterations, x.sub.n is the total number of nucleotides in SEQ ID NO: 1, and y is, for instance, 0.70 for 70%, 0.80 for 80%, 0.85 for 85%, 0.90 for 90%, 0.95 for 95%, etc., and wherein any non-integer product of x.sub.n and y is rounded down to the nearest integer prior to subtracting from x.sub.n.

Alterations of a polynucleotide sequence encoding the polypeptide of SEQ ID NO: 2 may create nonsense, missense or frameshift mutations in this coding sequence and thereby alter the polypeptide encoded by the polynucleotide following such alterations. Similarly, a polypeptide sequence may be identical to the reference sequence of SEQ ID NO: 2, that is be 100% identical, or it may include up to a certain integer number of amino acid alterations as compared to the reference sequence such that the percentage identity is less than 100%. Such alterations are selected from the group consisting of at least one amino acid deletion, substitution, including conservative and non-conservative substitution, or insertion, and wherein the alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in SEQ ID NO: 2 by the numerical percent of the respective percent identity (divided by 100) and then subtracting that product from the total number of amino acids in SEQ ID NO: 2, or:
n.sub.a.ltorsim.x.sub.a-(x.sub.a.y),
   wherein n.sub.a is the number of amino acid alterations, x.sub.a is the total number of amino acids in SEQ ID NO: 2, and y is, for instance 0.70 for 70%, 0.80 for 80%, 0.85 for 85% etc., and wherein any non-integer produce of x.sub.a and y is rounded down to the nearest integer prior to subtracting it from x.sub.a.

“Nucleic acids” are polymers of nucleotides, wherein a nucleotide comprises a base linked to a sugar which sugars are in turn linked one to another by an interceding at least bivalent molecule, such as phosphoric acid. In naturally occurring nucleic acids, the sugar is either 2′-deoxyribose (DNA) or ribose (RNA). Unnatural poly- or oliogonucleotides contain modified bases, sugars, or linking molecules, but are generally understood to mimic the complementary nature of the naturally occurring nucleic acids after which they are designed. An example of an unnatural oligonucleotide is an antisense molecule composition that has a phosphorothiorate backbone. An “oligonucleotide” generally refers to a nucleic acid molecule having less than 30 nucleotides.

A “polypeptide” is a polymer of amino acid residues joined by peptide bonds, and a peptide generally refers to amino acid polymers of 12 or less residues. Peptide bonds can be produced naturally as directed by the nucleic acid template or synthetically by methods well known in the art.

A “protein” is a macromolecule comprising one or more polypeptide chains. A protein may further comprise substituent groups attached to the side groups of the amino acids not involved in formation of the peptide bonds. Typically, proteins formed by eukaryotic cell expression also contain carbohydrates. Proteins are defined herein in terms of their amino acid sequence or backbone and substituents are not specified, whether known or not.

The term “receptor” denotes a molecule having biological activity resulting from interaction with a specific ligand or binding partner. Cell membrane bound receptors are characterized by an extracellular ligand-binding domain, one or more membrane spanning or transmembrane domains, and an intracellular effector domain that is typically involved in signal transduction. Ligand binding to cell membrane receptors causes changes in the extracellular domain that are communicated across the cell membrane, direct or indirect interaction with one or more intracellular proteins, and alters cellular properties, such as enzyme activity, cell shape, or gene expression profile. Receptors may also be untethered to the cell surface and may be cytosolic, nuclear, or released from the cell altogether. Non-cell associated receptors are termed soluble receptors.

All publications or patents cited herein are entirely incorporated herein by reference, whether or not specifically designated accordingly, as they show the state of the art at the time of the present invention and/or provide description and enablement of the present invention. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY (1987-2001); Sambrook, et al., Molecular Cloning: A Laboratory Manual, 2nd Edition, Cold Spring Harbor, N.Y. (1989); Harlow and Lane, antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY (1997-2001).

CNGH0011 proteins and nucleic acid molecules encoding them comprise a family of molecules having certain conserved structural and functional features. Each of these molecules is included in the invention. As used herein, the term “family” is intended to mean two or more proteins or nucleic acid molecules having a common or similar domain structure and having sufficient amino acid or nucleotide sequence identity as defined herein. Family members can be from either the same or different species. For example, a family can comprise two or more proteins of human origin, or can comprise one or more proteins of human origin and one or more of non-human origin.

A domain that may be present in CNGH0011 proteins is a signal sequence. As used herein, a “signal sequence” includes a peptide of at least about 10 amino acid residues in length which occurs at the amino terminus of membrane-bound proteins and which contains at least about 45% hydrophobic amino acid residues, such as alanine, leucine, isoleucine, phenylalanine, proline, tyrosine, tryptophan, or valine. In a preferred embodiment, a signal sequence contains at least about 10 to 35 amino acid residues, preferably about 10 to 20 amino acid residues, and has at least about 35-60%, more preferably 40-50%, and more preferably at least about 45% hydrophobic residues. A signal sequence serves to direct a protein containing such a sequence to a lipid bilayer. Thus, in one embodiment, a CNGH0011 protein may contain a signal sequence. The signal sequence is cleaved during processing of the mature protein.

CNGH0011 proteins can include an extracellular domain. As used herein, an “extracellular domain” refers to a portion of a protein that is localized to the non-cytoplasmic side of a lipid bilayer of a cell when a nucleic acid encoding the protein is expressed in the cell. The human CNGH0011 protein extracellular domain is located from about amino acid residues 33-46, 102-110, 192-210, and 263-350 of SEQ ID NO: 2.

In addition, CNGH0011 includes a transmembrane domain. As used herein, a “transmembrane domain” refers to an amino acid sequence which is at least about 15 amino acid residues in length and which contains at least about 65-70% hydrophobic amino acid residues such as alanine, leucine, phenylalanine, protein, tyrosine, tryptophan, or valine (Erik, et al. Proc. of Sixth Int. Conf. on Intelligent Systems for Molecular Biology, p 175-182). In a preferred embodiment, a transmembrane domain contains about 15-30 amino acid residues, preferably about 20-25 amino acid residues, and has at least about 60-80%, more preferably 65-75%, and more preferably at least about 70% hydrophobic residues. Thus, in one embodiment, a CNGH0011 protein of the invention contains one or more transmembrane domains corresponding to about amino acid residues 13-32, 47-66, 79-101, 111-133, 169-191, 211-233, and 240-262 of SEQ ID NO: 2. CNGH0011 can alternately exist in a membrane-bound form having at least one transmembrane domain of SEQ ID NO: 2.

The present invention includes CNGH0011 proteins having a cytoplasmic domain, particularly including proteins having a carboxyl-terminal cytoplasmic domain. As used herein, a “cytoplasmic domain” refers to a portion of a protein that is localized to the cytoplasmic side of a lipid bilayer of a cell when a nucleic acid encoding the protein is expressed in the cell. The human CNGH0011 cytoplasmic domain is situated from about amino acid residues 1-12, 67-78, 134-168, and 234-269 of SEQ ID NO: 2.

CNGH0011 proteins typically comprise a variety of potential post-translational modification sites (often within an extracellular domain).

I. Isolated Nucleic Acid Molecules

The invention includes nucleic acid molecules that encode a polypeptide of the invention (eg., SEQ ID NO:2). Such nucleic acids include, for example, the nucleotide sequence of SEQ ID NO: 1 and those with nucleotide sequence variants of SEQ ID NO: 1 or some portion thereof, such as the portion which encodes mature CNGH0011 protein, immature CNGH0011 protein, or a domain of CNGH0011 protein. These nucleic acids are collectively referred to as nucleic acids of the invention.

One aspect of the invention pertains to isolated nucleic acid molecules that encode a CNGH0011 polypeptide or a biologically active portion thereof, as well as nucleic acid molecules sufficient for use as hybridization probes to identify nucleic acid molecules encoding a CNGH0011 polypeptide and fragments of such nucleic acid molecules suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one that is separated from other nucleic acid molecules that are present in the natural source of the nucleic acid molecule. Preferably, an “isolated” nucleic acid molecule is free of sequences (preferably protein-encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A CNGH0011 nucleic acid molecule, a variant, or a complement thereof, can be isolated using standard molecular biology techniques and the sequence information provided herein. Using all or a portion of the nucleic acid sequence of SEQ ID NO: 1 or its variant as a hybridization probe, nucleic acid molecules of the invention can be isolated using standard hybridization and cloning techniques (e.g., as described in Sambrook et al., eds., Molecular Cloning: A Laboratory Manual, 2nd ed., Cold Spring Harbor Laboratory, Cold Spring Harbor Laboratory Press, Cold Spring Harbor, N.Y., 1989).

A nucleic acid molecule of the invention can be amplified using cDNA, mRNA or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which is a complement of a variant of the nucleotide sequence of SEQ ID NO: 1 or a portion thereof. A nucleic acid molecule which is complementary to a given nucleotide sequence is one which is sufficiently complementary to the given nucleotide sequence that it can hybridize to the given nucleotide sequence, thereby forming a stable duplex.

Moreover, a nucleic acid molecule of the invention can comprise only a portion of a nucleic acid sequence encoding a full length polypeptide of the invention for example, a fragment which can be used as a probe or primer or a fragment encoding a biologically active portion of a polypeptide of the invention. The nucleotide sequence determined from the cloning of a gene allows for the generation of probes and primers designed for use in identifying and/or cloning homologs in other cell types, e.g., from other tissues, as well as homologs from other mammals. The probe/primer typically comprises substantially purified oligonucleotide. The oligonucleotide typically comprises a region of nucleotide sequence that hybridizes under stringent conditions to at least about 15, preferably about 25, more preferably about 50, 75, 100, 125, 150, 175, 200, 250, 300, 350, or 400 or more consecutive nucleotides of the sense or anti-sense sequence of SEQ ID NO: 1 or of a naturally occurring mutant of SEQ ID NO: 1.

Probes based on the sequence of a nucleic acid molecule of the invention can be used to detect transcripts or genomic sequences encoding the same protein molecule encoded by a selected nucleic acid molecule. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

A nucleic acid fragment encoding a biologically active portion of a polypeptide of the invention can be prepared by isolating a portion of SEQ ID NO: 1, expressing the encoded portion of the polypeptide protein (e.g., by recombinant expression in vitro), and assessing the activity of the encoded portion of the polypeptide.

The invention further encompasses nucleic acid molecules that differ from the nucleotide sequence of SEQ ID NO: 1 due to degeneracy of the genetic code and thus encode the same protein as that encoded by the nucleotide sequence of SEQ ID NO: 1.

In addition to the nucleotide sequences of SEQ ID NO: 1, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes that occur alternatively at a given genetic locus.

As used herein, the phrase “allelic variant” refers to a nucleotide sequence that occurs at a given locus or to a polypeptide encoded by the nucleotide sequence.

As used herein, the terms “gene” and “recombinant gene” refer to nucleic acid molecules comprising an open reading frame encoding a polypeptide of the invention. Such natural allelic variations can typically result in 1-5% variance in the nucleotide sequence of a given gene. Alternative alleles can be identified by sequencing the gene of interest in a number of different individuals. This can be readily carried out using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

Moreover, nucleic acid molecules encoding proteins of the invention from other species (homologs) (for example, those disclosed in SEQ ID NOS: 3-6), which have a nucleotide sequence which differs from that of the protein described herein are intended to be within the scope of the invention. Nucleic acid molecules corresponding to natural allelic variants and homologs of a cDNA of the invention can be isolated based on their identity to human nucleic acid molecules using the cDNAs, or a portion thereof, as hybridization probes according to standard hybridization techniques under stringent hybridization conditions. For example, a cDNA encoding a soluble form of a membrane-bound protein of the invention can be isolated based on its hybridization to a nucleic acid molecule encoding all or part of the membrane-bound form. Likewise, a cDNA encoding a membrane-bound form can be isolated based on its hybridization to a nucleic acid molecule encoding all or part of the soluble form.

Accordingly, in another embodiment, an isolated nucleic acid molecule of the invention is at least 15 (25, 40, 60, 80, 100, 150, 200, 250, 300, 350, 400, 450, 550, 650, 700, 800, 900, 1000, 1100, 1200, 1300, 1400, 1500, 1600, 1700, 1800, 1900, 2000, 2100, or 2132) nucleotides in length and hybridizes under stringent conditions to the nucleic acid molecule comprising the nucleotide sequence, preferably the coding sequence, of SEQ ID NO: 1, or a complement thereof. As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions comprises hybridization in 6× sodium chloride/sodium citrate (SSC) at about 45° C., followed by one or more washes in 0.2×SSC, 0.1% SDS at about 50-65° C. Preferably, an isolated nucleic acid molecule of the invention that hybridizes under stringent conditions to the sequence of SEQ ID NO: 1, or a complement thereof, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of a nucleic acid molecule of the invention that can exist in the population, the skilled artisan will further appreciate that changes can be introduced by mutation thereby leading to changes in the amino acid sequence of the encoded protein, without altering the biological activity of the protein, including, additions, deletions, or substitutions. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a polypeptide of the invention that contain changes in amino acid residues that are not essential for activity. Such polypeptides differ in amino acid sequence from SEQ ID NO: 2, yet retain CNGH0011 biological activity. In one embodiment, the isolated nucleic acid molecule includes a nucleotide sequence encoding a CNGH0011 protein that includes an amino acid sequence that is at least about 40% identical, 50%, 60%, 70%, 80%, 90%, 95%, or 98% identical to the amino acid sequence of SEQ ID NO: 2.

An isolated nucleic acid molecule encoding a variant protein can be created by introducing one or more nucleotide substitutions, additions or deletions into the nucleotide sequence of SEQ ID NO: 1, such that one or more amino acid residue substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

In a preferred embodiment, a mutant polypeptide that is a variant of a polypeptide of the invention can be assayed for: (1) the ability to form protein:protein interactions with the polypeptide of the invention; (2) the ability to bind a ligand of the polypeptide of the invention (e.g., another protein identified herein); (3) the ability to bind to a modulator or substrate of the polypeptide of the invention; or (4) the ability to modulate a physiological activity of the protein, such as one of those disclosed herein.

The present invention encompasses antisense nucleic acid molecules, i.e., molecules that are complementary to a sense nucleic acid encoding a polypeptide of the invention, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid molecule can be antisense to all or part of a non-coding region of the coding strand of a nucleotide sequence encoding a polypeptide of the invention. The non-coding regions (“5′ and 3“untranslated regions”) are the 5′ and 3′ sequences that flank the coding region and are not translated into amino acids.

An antisense oligonucleotide can be, for example, about 5, 10, 15, 20, 25, 30, 35, 40, 45, or 50 or more nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine, as well as shown in the table below. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a selected polypeptide of the invention to thereby inhibit expression, e.g., by inhibiting transcription and/or translation. The hybridization can be by conventional nucleotide complementarity to form a stable duplex or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual α-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The invention also encompasses ribozymes. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach (1988) Nature 334:585-591) can be used to catalytically cleave mRNA transcripts to thereby inhibit translation of the protein encoded by the mRNA. A ribozyme having specificity for a nucleic acid molecule encoding a polypeptide of the invention can be designed based upon the nucleotide sequence of a cDNA disclosed herein. For example, a derivative of a Tetrahymena L-19 IVS RNA can be constructed in which the nucleotide sequence of the active site is complementary to the nucleotide sequence to be cleaved in Cech et al., U.S. Pat. No. 4,987,071; and Cech et al., U.S. Pat. No. 5,116,742. Alternatively, an mRNA encoding a polypeptide of the invention can be used to select a catalytic RNA having a specific ribonuclease activity from a pool of RNA molecules. See, e.g., Bartel and Szostak (1993) Science 261:1411-1418.

The invention also encompasses ribonucleic acid molecules which are complementary, antisense, double stranded homologues, siRNA, or are sequence specific single-stranded RNAs which form short hairpin structures, shRNA (collectively, interfering RNA), that can be used to down-modulate specific gene expression, in this case, CNGH0011, and therefore to inhibit protein expression and to elucidate their respective biological functions. (Fire, A., et al. (1998) Nature 391: 806-811; Paddison, P. J. et al. (2002) Genes Develop 16:948-958).

The invention further encompasses aptamer molecules that modulate activity and/or expression of the CNGH0011 protein. Aptamers are DNA or RNA molecules or hybrids thereof that are designed to bind to a molecule, ligand, or receptor to affect its expression or activity (e.g., their three-dimensional structure enables them to bind to a certain place on the molecule whose activity is being affected).

In various embodiments, the nucleic acid molecules of the invention can be modified at the base moiety, sugar moiety, or phosphate backbone to improve, e.g., the stability, hybridization, or solubility of the molecule. For example, the nucleotide analogs shown in the table below and as described above, can be substituted for the naturally occurring nucleotides.

| Nucleotide Analog | Symbol |
|---|---|
| 4-acetylcytidine | ac4c |
| 5-(carboxyhydroxymethyl)uridine | chm5u |
| 2′-O-methylcytidine | Cm |
| 5-carboxymethylaminomethyl-2-thiouridine | cmnm5s2u |
| 5-carboxymethylaminomethyluridine | cmnm5u |
| Dihydrouridine | D |
| 2′-O-methylpseudouridine | Fm |
| beta, D-galactosylqueuosine | gal q |
| 2′-O-methylguanosine | Gm |
| Inosine | I |
| N6-isopentenyladenosine | i6a |
| 1-methyladenosine | m1a |
| 1-methylpseudouridine | m1f |
| 1-methylguanosine | m1g |
| 1-methylinosine | m1i |
| 2,2-dimethylguanosine | m22g |
| 2-methyladenosine | m2a |
| 2-methylguanosine | m2g |
| 3-methylcytidine | m3c |
| 5-methylcytidine | m5c |
| N6-methyladenosine | m6a |
| 7-methylguanosine | m7g |
| 5-methylaminomethyluridine | Mam5u |
| 5-methoxyaminomethyl-2-thiouridine | Mam5s2u |
| beta, D-mannosylqueuosine | Man q |
| 5-methoxycarbonylmethyl-2-thiouridine | Mcm5s2u |
| 5-methoxycarbonylmethyluridine | Mcm5u |
| 5-methoxyuridine | mo5u |
| 2-methylthio-N6-isopentenyladenosine | ms2i6a |
| N-((9-beta-D-ribofuranosyl-2-methylthiopurine-6- | ms2t6a |
| yl)carbamoyl)threonine | |
| N-((9-beta-D-ribofuranosylpurine-6-yl)N- | mt6a |
| methylcarbamoyl)threonine | |
| Uridine-5-oxyacetic acid-methylester | Mv |
| Uridine-5-oxyacetic acid | o5u |
| Wybutoxosine | osyw |
| Pseudouridine | P |
| Queuosine | Q |
| 5-methyl-2-thiouridine | s2t |
| 2-thiocytidine | s2c |
| 5-methyl-2-thiouridine | s2t |
| 2-thiouridine | s2u |
| 4-thiouridine | s4u |
| 5-methyluridine | T |
| N-((9-beta-D-ribofuranosylpurine-6-yl)-carbamoyl)threonine | t6a |
| 2′-O-methyl-5-methyluridine | Tm |
| 2′-O-methyluridine | Um |
| Wybutosine | Yw |
| 3-(3-amino-3-carboxy-propyl)uridine, (acp3)u | X |

In another example, the deoxyribose phosphate backbone of the nucleic acids can be modified to generate peptide nucleic acids (see Hyrup et al. (1996) Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93: 14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup (1996), supra; Perry-O'Keefe et al. (1996) Proc. Natl. Acad. Sci. USA 93: 14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996), supra). The synthesis of PNA-DNA chimeras can be performed as described in Hyrup (1996), supra, and Finn et al. (1996) Nucleic Acids Res. 24(17): 3357-63. For example, a DNA chain can be synthesized on a solid support using standard phosphoramidite coupling chemistry and modified nucleoside analogs. Compounds, such as 5′-(4-methoxytrityl)amino-5′-deoxy-thymidine phosphoramidite, can be used as a link between the PNA and the 5′ end of DNA (Mag et al. (1989) Nucleic Acids Res. 17:5973-88). PNA monomers are then coupled in a step-wise manner to produce a chimeric molecule with a 5′ PNA segment and a 3′ DNA segment (Finn et al. (1996) Nucleic Acids Res. 24(17):3357-63). Alternatively, chimeric molecules can be synthesized with a 5′ DNA segment and a 3′ PNA segment (Peterser et al. (1975) Bioorganic Med. Chem. Lett. 5:1119-11124).

In other embodiments, the oligonucleotide can include other appended groups, such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the oligonucleotide can be conjugated to another molecule, e.g., a peptide, hybridization triggered cross-linking agent, transport agent, hybridization-triggered cleavage agent, etc.

II. Isolated Proteins and Antibodies

A. Proteins

The invention thus includes purified human CNGH0011 protein, both in the form of the immature amino acid residue protein and in the form of the mature protein. Mature human CNGH0011 protein can be synthesized without the signal sequence polypeptide at the amino terminus thereof, or it can be synthesized by generating immature CNGH0011 protein and cleaving the signal sequence therefrom.

It is furthermore recognized that CNGH0011 can exist, in a membrane bound form, wherein the protein has at least one transmembrane region.

In addition to full length mature and immature human CNGH0011 proteins, the invention includes fragments, derivatives, and variants of these CNGH0011 proteins, as described herein. These proteins, fragments, derivatives, and variants are collectively referred to herein as polypeptides of the invention or proteins of the invention.

One aspect of the invention pertains to isolated proteins, and biologically active portions thereof, as well as polypeptide fragments suitable for use as immunogens to raise antibodies directed against a polypeptide of the invention. In one embodiment, the native polypeptide can be isolated from cells or tissue sources by an appropriate purification scheme using standard protein purification techniques. In another embodiment, polypeptides of the invention are produced by recombinant DNA techniques. Alternative to recombinant expression, a polypeptide of the invention can be synthesized chemically using standard peptide synthesis techniques, e.g., standard Boc or FMOC chemistry. The resulting peptides can be injected as is, cross-linked, or conjugated to a carrier molecule. To facilitate conjugation to carriers, an N-terminal N-acetyl-cysteine or C-terminal amides can be formed and the C-terminal amino acid can be amidated. A variety of linking groups may be interspaced between the peptide and the carrier molecule to allow for proper conformational folding and presentation of the antigenic peptide.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals that are involved in the synthesis of the protein. Accordingly such preparations of the protein have less than about 30%, 20%, 10%, 5% (by dry weight) of chemical precursors or compounds other than the polypeptide of interest.

Biologically active portions of a polypeptide of the invention include polypeptides comprising amino acid sequences sufficiently identical to or derived from the amino acid sequence of the protein (e.g., the amino acid sequence shown in SEQ ID NO: 2), which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide that is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

Preferred polypeptides are substantially identical (e.g., at least about 40%, preferably 50%, 60%, 70%, 80%, 90%, 95%, or 99%) to SEQ ID NO: 2 and retain the functional activity of the corresponding naturally-occurring CNGH0011 protein yet differ in amino acid sequence due to natural allelic variation or mutagenesis.

The invention also provides chimeric or fusion proteins. As used herein, a “chimeric protein” or “fusion protein” comprises all or part (preferably biologically active) of a CNGH0011 polypeptide operably linked to a heterologous polypeptide (i.e., a polypeptide other than the same polypeptide of the invention). Within the fusion protein, the term “operably linked” is intended to indicate that the CNGH0011 polypeptide and the heterologous polypeptide are fused in-frame to each other. The heterologous polypeptide can be fused to the amino-terminus or the carboxyl-terminus of the CNGH0011 polypeptide. In another embodiment, a CNGH0011 polypeptide or a domain or active fragment thereof can be fused with a heterologous protein sequence or fragment thereof to form a chimeric protein, where the polypeptides, domains or fragments are not fused end to end but are interposed within the heterologous protein framework.

One useful fusion protein is a GST fusion protein in which the CNGH0011 polypeptide is fused to the carboxyl terminus of GST sequences. Such fusion proteins can facilitate the purification of a recombinant CNGH0011 polypeptide.

In another embodiment, the fusion protein contains a heterologous signal sequence at its amino terminus. For example, the native signal sequence of a CNGH0011 polypeptide can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a CNGH0011 polypeptide is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a CNGH0011 polypeptide. Inhibition of ligand/receptor interaction can be useful therapeutically, both for treating proliferative and differentiative disorders and for modulating (e.g., promoting or inhibiting) cell survival. A preferred embodiment of an immunoglobulin chimeric protein is a CH1 domain-deleted immunoglobulin or “mimetibody” having an active polypeptide fragment interposed within a modified framework region as taught in co-pending application PCT WO/04002417. Moreover, the immunoglobulin fusion proteins of the invention can be used as immunogens to produce antibodies directed against a CNGH0011 polypeptide in a subject, to purify ligands and in screening assays to identify molecules that inhibit the interaction of receptors with ligands.

Chimeric and fusion proteins of the invention can be produced by standard recombinant DNA techniques. In another embodiment, the fusion gene can be synthesized by conventional techniques, including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a CNGH0011 polypeptide can be cloned into such an expression vector such that the fusion moiety is linked in-frame to the CNGH0011 polypeptide.

A signal sequence of a CNGH0011 polypeptide (e.g., the signal sequence in SEQ ID NO: 2) can be used to facilitate secretion and isolation of the secreted protein or other proteins of interest. Signal sequences are typically characterized by a core of hydrophobic amino acids that are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to the signal sequence itself and to the polypeptide in the absence of the signal sequence (i.e., the cleavage products). In one embodiment, a nucleic acid sequence encoding a signal sequence of the invention can be operably linked in an expression vector to a protein of interest, such as a protein that is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence that facilitates purification, such as with a GST domain.

In another embodiment, the signal sequences of the present invention can be used to identify regulatory sequences, e.g., promoters, enhancers, and/or repressors. Since signal sequences are the most amino-terminal sequences of a peptide, the nucleic acids flanking the signal sequence on its amino-terminal side are likely regulatory sequences that affect transcription. Thus, a nucleotide sequence that encodes all or a portion of a signal sequence can be used as a probe to identify and isolate signal sequences and their flanking regions, and these flanking regions can be studied to identify regulatory elements therein.

The present invention also pertains to variants of the CNGH0011 polypeptides and can include one or more amino acid substitutions, deletions or additions, either from natural mutations or human manipulation, as specified herein. Such mutations or substitutions can include muteins, whose mutations can be significant enough to alter the properties of the peptide without altering the biological activity of the peptide to inhibit the binding of human CNGH0011 to its ligand. Of course, the number of amino acid substitutions a skilled artisan would make depends on many factors, including those described above. In certain embodiments of the invention, the number of amino acid substitutions, insertions or deletions for any given CNGH0011 polypeptide, fragment or variant will not be more than 1-5, or any range or value therein, as specified herein.

The polypeptides of the invention may also comprise modified, non-naturally occurring and unusual amino acids substituted or added to their amino acid sequences. A list of exemplary modified, non-naturally occurring and unusual amino acids is provided below.

| Modified (Unusual) Amino Acid | Symbol |
|---|---|
| 2-Aminoadipic acid | Aad |
| 3-Aminoadipic acid | Baad |
| beta-Alanine, beta-Aminopropionic acid | bAla |
| 2-Aminobutyric acid | Abu |
| 4-Aminobutyric acid, piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | BAib |
| 2-Aminopimelic acid | Apm |
| 2,4-Diaminobutyric acid | Dbu |
| Desmosine | Des |
| 2,2′-Diaminopimelic acid | Dpm |
| 2,3-Diaminopropionic acid | Dpr |
| N-Ethylglycine | EtGly |
| N-Ethylasparagine | EtAsn |
| Hydroxylysine | Hyl |
| allo-Hydroxylysine | AHyl |
| 3-Hydroxyproline | 3Hyp |
| 4-Hydroxyproline | 4Hyp |
| Isodesmosine | Ide |
| allo-Isoleucine | AIle |
| N-Methylglycine, sarcosine | MeGly |
| N-Methylisoleucine | MeIle |
| 6-N-Methyllysine | MeLys |
| N-Methylvaline | MeVal |
| Norvaline | Nva |
| Norleucine | Nle |
| Ornithine | Orn |

Amino acids in a CNGH0011 peptide of the present invention that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to, at least one CNGH0011 neutralizing activity.

Such variants have an altered amino acid sequence and can function as either agonists (mimetics) or as antagonists. Variants can be generated by mutagenesis, e.g., discrete point mutation or truncation. An agonist, for example, can retain substantially the same, or a subset, of the biological activities of the naturally occurring form of the protein, and/or can be a molecule that increases the level of expression of the protein. An antagonist of a protein can inhibit one or more of the activities of the naturally occurring form of the protein directly or indirectly by, for example, reducing the level of expression (e.g., with siRNA) or competitively binding to a downstream or upstream member of a cellular signaling cascade that includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention that function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods that can be used to produce libraries of potential variants of the CNGH0011 polypeptides from a degenerate oligonucleotide sequence. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of the coding sequence of a CNGH0011 polypeptide can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan (1992) Proc. Natl. Acad. Sci. USA 89:7811-7815; Delgrave et al. (1993) Protein Engineering 6(3):327-331).

B. Antibodies

The present invention further includes, but is not limited to, methods of using the nucleic acids and polypeptides encoded thereby to make antibodies and anti-idiotype antibodies, including diagnostic and therapeutic compositions, methods and devices. Such antibodies optionally further affect a specific ligand, such as but not limited to, where such antibody modulates, decreases, increases, antagonizes, agonizes, mitigates, alleviates, blocks, inhibits, abrogates and/or interferes with at least one CNGH0011 activity or binding, or with CNGH0011 receptor activity or binding, in vitro, in situ and/or in vivo. As a non-limiting example, a suitable CNGH0011 antibody, specified portion or variant of the present invention can bind at least one CNGH0011 protein, or specified portions, variants or domains thereof. A suitable CNGH0011 antibody, specified portion, or variant can also optionally affect at least one of CNGH0011 activity or function, such as but not limited to, RNA, DNA or polypeptide synthesis, CNGH0011 release, CNGH0011 receptor signaling, membrane CNGH0011 cleavage, CNGH0011 activity, CNGH0011 production and/or synthesis. CNGH0011 antibodies useful in the methods and compositions of the present invention can optionally be characterized by high affinity binding to CNGH0011 and optionally and preferably having low toxicity

As used herein, a “CNGH0011 antibody,” and the like include any polypeptide or peptide containing molecule that comprises at least a portion of an immunoglobulin molecule, such as but not limited to, at least one complementarity determinng region (CDR) of a heavy or light chain or a ligand binding portion thereof, a heavy chain or light chain variable region, a heavy chain or light chain constant region, a framework region, or any portion, fragment or variant thereof, or at least one portion of a CNGH0011 receptor or binding polypeptide, which can be incorporated into a CNGH0011 antibody of the present invention.

Antibodies can include one or more of at least one CDR, at least one variable region, at least one constant region, at least one heavy chain (e.g., g1, g2, g3, g4, m, a1, a2, d, e), at least one light chain (e.g., kappa (k) and lambda (1)), or any portion or fragment thereof, and can further comprise interchain and intrachain disulfide bonds, hinge regions, glycosylation sites that can be separated by a hinge region, as well as heavy chains and light chains. Light chains typically have a molecular weight of about 25 Kd and heavy chains typically range from about 50K-77 Kd. Light chains can exist in two distinct forms or isotypes, k and 1, which can combine with any of the heavy chain types. All light chains have at least one variable region and at least one constant region. The IgG antibody is considered a typical antibody structure and has two intrachain disulfide bonds in the light chain (one in the variable region and one in the constant region), with four in the heavy chain, and such bond encompassing a peptide loop of about 60-70 amino acids comprising a “domain” of about 110 amino acids in the chain. IgG antibodies can be characterized into four classes, IgG1, IgG2, IgG3 and IgG4. Each immunoglobulin class has a different set of functions. The following table summarizes the Physicochemical properties of each of the immunoglobulin classes and subclasses.

| Property | IgG1 | IgG2 | IgG3 | IgG4 | IgM | IgA1 | IgA2 | SigA | IgD | IgE |
|---|---|---|---|---|---|---|---|---|---|---|
| Heavy Chain | γ1 | γ1 | γ1 | γ1 | μ | α1 | α2 | α1/α2 | δ | e |
| Mean Serum | 9 | 3 | 1 | 0.5 | 1.5 | 3.0 | 0.5 | 0.05 | 0.03 | 0.00005 |
| conc. (mg/ml) | | | | | | | | | | |
| Sedimentation | 7 s | 7 s | 7 s | 7 s | 19 s | 7 s | 7 s | 11 s | 7 s | 8 s |
| constant | | | | | | | | | | |
| Mol. Wt. (×10³) | 146 | 146 | 170 | 146 | 970 | 160 | 160 | 385 | 184 | 188 |
| Half Life (days) | 21 | 20 | 7 | 21 | 10 | 6 | 6 | ? | 3 | 2 |
| % intravascular | 45 | 45 | 45 | 45 | 80 | 42 | 42 | Trace | 75 | 50 |
| distribution | | | | | | | | | | |
| Carbohydrate | 2-3 | 2-3 | 2-3 | 2-3 | 12 | 7-11 | 7-11 | 7-11 | 9-14 | 12 |
| (%) | | | | | | | | | | |

The following table summarizes non-limiting examples of antibody effector functions for human antibody classes and subclasses.

| Effector | | | | | | | | |
|---|---|---|---|---|---|---|---|---|
| function | IgG1 | IgG2 | IgG3 | IgG4 | IgM | IgA | IgD | IgE |
| Complement | + | +/− | ++ | − | ++ | − | − | − |
| fixation | | | | | | | | |
| Placental | + | +/− | + | + | − | − | − | − |
| transfer | | | | | | | | |
| Binding to | +++ | +++ | − | +++ | − | − | − | − |
| Staph A | | | | | | | | |
| Binding to | +++ | +++ | +++ | +++ | − | − | − | − |
| Strep G | | | | | | | | |

+++ = very high;
++ = high;
+ = moderate;
+/− = minimal;
− = none;
? = questionable

Accordingly, the type of antibody or fragment thereof can be selected for use according to the present invention based on the desired characteristics and functions that are desired for a particular therapeutic or diagnostic use, such as but not limited to, serum half life, intravascular distribution, complement fixation, etc.

The isolated CNGH0011 nucleic acids can be used for production of at least one CNGH0011 antibody or specified variant thereof, which can be used to measure or effect in a cell, tissue, organ or animal (including mammals and humans), to diagnose, monitor, modulate, treat, alleviate, help prevent the incidence of, or reduce the symptoms of, at least one CNGH0011 condition, selected from, but not limited to, at least one of an immune disorder or disease, a cardiovascular disorder or disease, an infectious, malignant, and/or neurologic disorder or disease, or other known or specified CNGH0011-related condition. A CNGH0011-related condition includes, without limitation, immune-mediated inflammatory diseases, such as COPD (asthma, emphysema, etc.).

Such a method can comprise administering an effective amount of a composition or a pharmaceutical composition comprising at least one CNGH0011 antibody to a cell, tissue, organ, animal or patient in need of such modulation, treatment, alleviation, prevention, or reduction in symptoms, effects or mechanisms. The effective amount can comprise an amount of about 0.001 to about 500 mg/kg per single (e.g., bolus), multiple or continuous administration, or to achieve a serum concentration of 0.01-5000 mg/ml serum concentration per single, multiple, or continuous administration, or any effective range or value therein, as done and determined using known methods, as described herein or known in the relevant arts.

An isolated CNGH0011 polypeptide, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a CNGH0011 protein comprises at least 8 (preferably 10, 15, 20, or 30 or more) amino acid residues of the amino acid sequence of SEQ ID NO: 2 and encompasses an epitope of the protein such that an antibody raised against the peptide forms a specific immune complex with the protein.

An immunogen typically is used to prepare antibodies by immunizing a suitable (i.e., immunocompetent) subject, such as a rabbit, goat, mouse, or other mammal or vertebrate. An appropriate immunogenic preparation can contain, for example, recombinantly expressed or chemically synthesized polypeptide. The preparation can further include an adjuvant, such as Freund's complete or incomplete adjuvant, or a similar immunostimulatory agent.

Antibody-producing cells can be obtained from the peripheral blood or, preferably, the spleen or lymph nodes of humans or other suitable animals that have been immunized with the immunogen of interest. Any other suitable host cell can also be used for expressing heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods. Cells that produce antibodies with the desired specificity can be selected by a suitable assay (e.g., ELISA).

In one approach, a hybridoma is produced by fusing a suitable immortal cell line (e.g., a myeloma cell line, such as, but not limited to, Sp2/0, Sp2/0-AG14, NSO, NS1, NS2, AE-1, L.5, >243, P3X63Ag8.653, Sp2 SA3, Sp2 MAI, Sp2 SS1, Sp2 SA5, U937, MLA 144, ACT IV, MOLT4, DA-1, JURKAT, WEHI, K-562, COS, RAJI, NIH 3T3, HL-60, MLA 144, NAMALWA, NEURO 2A, or the like), or heteromyelomas, fusion products thereof, or any cell or fusion cell derived therefrom, or any other suitable cell line as known in the art (see, e.g., www.atcc.org, www.lifetech.com, and the like), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. See, e.g., Ausubel, supra, and Colligan, Immunology, supra, chapter 2, entirely incorporated herein by reference.

Other suitable methods of producing or isolating antibodies of the requisite specificity can be used, including, but not limited to, methods that select recombinant antibody from a peptide or polypeptide library (e.g., but not limited to, a bacteriophage, ribosome, oligonucleotide, RNA, cDNA, or the like, display library; e.g., as available from Cambridge antibody Technologies, Cambridgeshire, UK; MorphoSys, Martinsreid/Planegg, DE; Biovation, Aberdeen, Scotland, UK; BioInvent, Lund, Sweden; Dyax Corp., Enzon, Affymax/Biosite; Xoma, Berkeley, Calif.; Ixsys. See, e.g., EP Publication No. 368,684, PCT/GB91/01134; PCT/GB92/01755; PCT/GB92/002240; PCT/GB92/00883; PCT/GB93/00605; U.S. Pat. No. 5,962,255; PCT/GB94/01422; PCT/GB94/02662; PCT/GB97/01835; (CAT/MRC); WO90/14443; WO90/14424; WO90/14430; PCT/US94/1234; WO92/18619; WO96/07754; (Scripps); EP 614 989 (MorphoSys); WO95/16027 (BioInvent); WO88/06630; WO90/3809 (Dyax); U.S. Pat. No. 4,704,692 (Enzon); PCT/US91/02989 (Affymax); WO89/06283; EP 371 998; EP 550 400; (Xoma); EP 229 046; PCT/US91/07149 (Ixsys); or stochastically generated peptides or polypeptides—U.S. Pat. Nos. 5,723,323, 5763192, 5814476, 5817483, 5824514, and 5976862, WO 86/05803, EP 590 689 (Ixsys, now Applied Molecular Evolution (AME), each entirely incorporated herein by reference) or that rely upon immunization of transgenic animals (e.g., SCID mice, Nguyen et al., Microbiol. Immunol. 41:901-907 (1997); Sandhu et al., Crit. Rev. Biotechnol. 16:95-118 (1996); Eren et al., Immunol. 93:154-161 (1998), each entirely incorporated by reference as well as related patents and applications) that are capable of producing a repertoire of human antibodies, as known in the art and/or as described herein. Such techniques, include, but are not limited to, ribosome display (Hanes et al., Proc. Natl. Acad. Sci. USA, 94:4937-4942 (May 1997); Hanes et al., Proc. Natl. Acad. Sci. USA, 95:14130-14135 (Nov. 1998)); single cell antibody producing technologies (e.g., selected lymphocyte antibody method (“SLAM”) (U.S. Pat. No. 5,627,052, Wen et al., J. Immunol. 17:887-892 (1987); Babcook et al., Proc. Natl. Acad. Sci. USA 93:7843-7848 (1996)); gel microdroplet and flow cytometry (Powell et al., Biotechnol. 8:333-337 (1990); One Cell Systems, Cambridge, Mass.; Gray et al., J. Imm. Meth. 182:155-163 (1995); Kenny et al., Bio/Technol. 13:787-790 (1995)); B-cell selection (Steenbakkers et al., Molec. Biol. Reports 19:125-134 (1994); Jonak et al., Progress Biotech, Vol. 5, In Vitro Immunization in Hybridoma Technology, Borrebaeck, ed., Elsevier Science Publishers B.V., Amsterdam, Netherlands (1988)).

Methods for engineering or humanizing non-human or human antibodies can also be used and are well known in the art. Generally, a humanized or engineered antibody has one or more amino acid residues from a source that is non-human, e.g., but not limited to, mouse, rat, rabbit, non-human primate or other mammal in addition to human amino acid residues. These human amino acid residues are often referred to as “import” residues, which are typically taken from an “import” variable, constant or other domain of a known human sequence. Known human Ig sequences are disclosed, e.g., www.ncbi.nlm.nih.gov/entrez/query.fcgi; www.atcc.org/phage/hdb.html; www.sciquest.com/; www.abcam.com; www.antibodyresource.com/onlinecomp.html; www.public.iastate.edu/˜pedro/research_tools.html; www.mgen.uni-heidelberg.de/SD/IT/IT.html; www.whfreeman.com/immunology/CH05/kuby05.htm; www.library.thinkquest.org/12429/Immune/Antibody.html; www.hhmi.org/grants/lectures/1996/vlab/; www.path.cam.ac.uk/˜mrc7/mikeimages.html; www.antibodyresource.com/; mcb.harvard.edu/BioLinks/Immunology.html. www.immunologylink.com/; pathbox.wustl.edu/˜hcenter/index.html; www.biotech.ufl.edu/˜hcl/; www.pebio.con/pa/340913/340913.html; www.nal.usda.gov/awic/pubs/antibody/; www.m.ehime-u.ac.jp/˜yasuhito/Elisa.html; www.biodesign.com/table.asp; www.icnet.uk/axp/facs/davies/links.html; www.biotech.ufl.edu/˜fccl/protocol.html; www.isac-net.org/sites_geo.htmi; aximt1.imt.uni-marburg.de/˜rek/AEPStart.html; baserv.uci.kun.nl/˜jraatsAinksl.html; www.recab.uni-hd.de/immuno.bme.nwu.edu/; www.mrc-cpe.cam.ac.uk/imt-doc/public/INTRO.html; www.ibt.unam.mx/vir/V_mice.html; imgt.cnusc.fr:8104/; www.biochem.ucl.ac.uk/˜martin/abs/index.html; antibody.bath.ac.uk/; abgen.cvm.tamu.edu/ab/wwwabgen.html; www.unizh.ch/˜honegger/AHOseminar/Slide01.html; www.cryst.bbk.ac.uk/˜ubcg07s/; www.nimr.mrc.ac.uk/CC/ccaewg/ccaewg.htm; www.path.cam.ac.uk/˜mrc7/humanisation/TAHHP.html; www.ibt.unam.mx/vir/structure/stat_aim.html; www.biosci.missouri.edu/smithgp/index.html; www.cryst.bioc.cam.ac.uk/˜fmolina/Web-pages/Pept/spottech.html; www.jerini.de/fr_products.htm; www.patents.ibm.com/ibm.html.Kabat et al., Sequences of Polypeptides of Immunological Interest, U.S. Dept. Health (1983), each entirely incorporated herein by reference.

Such imported sequences can be used to reduce immunogenicity or reduce, enhance or modify binding, affinity, on-rate, off-rate, avidity, specificity, half-life, or any other suitable characteristic, as known in the art. Generally, part or all of the non-human or human CDR sequences are maintained while the non-human sequences of the variable and constant regions are replaced with human or other amino acids. Antibodies can also optionally be humanized with retention of high affinity for the antigen and other favorable biological properties. To achieve this goal, humanized antibodies can be optionally prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences (e.g., from known libraries) so that the desired antibody characteristic, such as increased affinity for the target antigen(s), is achieved. In general, the CDR residues are directly and most substantially involved in influencing antigen binding. Humanization or engineering of antibodies of the present invention can be performed using any known method, such as but not limited to, those described in, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567; PCT/: US98/16280; US96/18978; US91/09630; US91/05939; US94/01234; GB89/01334; GB91/01134; GB92/01755; WO90/14443; WO90/14424; and WO90/14430; EP 229246; each entirely incorporated herein by reference, including references cited therein.

The CNGH0011 antibody can also be optionally generated by immunization of a transgenic animal (e.g., mouse, rat, hamster, non-human primate, and the like) capable of producing a repertoire of human antibodies, as described herein and/or as known in the art. Cells that produce a human CNGH0011 antibody can be isolated from such animals and immortalized using suitable methods, such as the methods described herein.

Transgenic mice that can produce a repertoire of human antibodies that bind to human antigens can be produced by known methods (e.g., but not limited to, U.S. Pat. Nos. 5,770,428, 5,569,825, 5,545,806, 5,625,126, 5,625,825, 5,633,425, 5,661,016 and 5,789,650 issued to Lonberg et al.; Jakobovits et al. WO 98/50433, Jakobovits et al. WO 98/24893, Lonberg et al. WO 98/24884, Lonberg et al. WO 97/13852, Lonberg et al. WO 94/25585, Kucherlapate et al. WO 96/34096, Kucherlapate et al. EP0463 151 B1, Kucherlapate et al. EP0710719 A1, Surani et al. U.S. Pat. No. 5,545,807, Bruggemann et al. WO 90/04036, Bruggemann et al. EP 0438 474 B1, Lonberg et al. EP 0814 259 A2, Lonberg et al. GB 2 272 440 A, Lonberg et al. Nature 368:856-859 (1994), Taylor et al., Int. Immunol. 6(4)579-591 (1994), Green et al, Nature Genetics 7:13-21 (1994), Mendez et al., Nature Genetics 15:146-156 (1997), Taylor et al., Nucleic Acids Research 20(23):6287-6295 (1992), Tuaillon et al., Proc Natl Acad Sci USA 90(8)3720-3724 (1993), Lonberg et al., Int Rev Immunol 13(1):65-93 (1995) and Fishwald et al., Nat Biotechnol 14(7):845-851 (1996), which are each entirely incorporated herein by reference). Generally, these mice comprise at least one transgene comprising DNA from at least one human immunoglobulin locus that is functionally rearranged, or which can undergo functional rearrangement. The endogenous immunoglobulin loci in such mice can be disrupted or deleted to eliminate the capacity of the animal to produce antibodies encoded by endogenous genes.

Antibodies of the present invention can also be prepared in milk by administering at least one anti-CNGH0011 antibody encoding nucleic acid to transgenic animals or mammals, such as goats, cows, horses, rabbits, sheep, and the like, that produce antibodies in their milk. Such animals can be provided using known methods. See, e.g., but not limited to, U.S. Pat. Nos. 5,827,690; 5,849,992; 4,873,316; 5,849,992; 5,994,616; 5,565,362; 5,304,489, and the like, each of which is entirely incorporated herein by reference. Antibodies of the present invention can additionally be prepared using at least one CNGH0011 antibody encoding nucleic acid to provide transgenic plants and cultured plant cells (e.g., but not limited to, tobacco and maize) that produce such antibodies, specified portions or variants in the plant parts or in cells cultured therefrom.

The antibodies of the invention can bind human CNGH0011 with a wide range of affinities (K_{D}). In a preferred embodiment, at least one human mAb of the present invention can optionally bind human CNGH0011 with high affinity. For example, a human mAb can bind human CNGH0011 with a K_{D} equal to or less than about 10⁻⁷ M, such as but not limited to, 0.1-9.9 (or any range or value therein) X 10⁻⁷, 10⁻⁸, 10⁻⁹,10⁻¹⁰, 10⁻¹¹, 10⁻¹², 10⁻¹³ or any range or value therein.

The affinity or avidity of an antibody for an antigen can be determined experimentally using any suitable method. (See, for example, Berzofsky, et al., “Antibody-Antigen Interactions,” In Fundamental Immunology, Paul, W. E., Ed., Raven Press: New York, N.Y. (1984); Kuby, Janis Immunology, W.H. Freeman and Company: New York, N.Y. (1992); and methods described herein). The measured affinity of a particular antibody-antigen interaction can vary if measured under different conditions (e.g., salt concentration, pH). Thus, measurements of affinity and other antigen-binding parameters (e.g., K_{D}, Kₒₙ, K_{off}) are preferably made with standardized solutions of antibody and antigen, and a standardized buffer, such as the buffer described herein.

An antibody directed against a polypeptide of the invention (e.g., monoclonal antibody) can be used to isolate the polypeptide by standard techniques, such as affinity chromatography or immunoprecipitation. Moreover, such an antibody can be used to detect the protein (e.g., in a cellular lysate or cell supernatant) in order to evaluate the abundance and pattern of expression of the polypeptide. The antibodies can also be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, preferably expression vectors, containing a nucleic acid encoding a CNGH0011 polypeptide (or a portion thereof). As used herein, the term vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion and chimeric proteins or peptides, encoded by nucleic acids as described herein.

The recombinant expression vectors of the invention can be designed for expression of a polypeptide of the invention in prokaryotic (e.g., E. coli) or eukaryotic cells (e.g., insect cells (using baculovirus expression vectors), yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. To assist in affinity purification, various tag polypeptides and their respective antibodies are well known in the art. Examples include poly-histidine (poly-his) or poly-histidine-glycine (poly-his-gly) tags; the flu HA tag polypeptide and its antibody 12CA5 (Field et al., Mol. Cell. Biol, 8:2159-2165 (1988)); the c-myc tag and the 8F9, 3C7, 6E10, G4, B7 and 9E10 antibodies thereto (Evan et al., Molecular and Cellular Biology, 5:3610-3616 (1985)); and the Herpes Simplex virus glycoprotein D (gD) tag and its antibody (Paborsky et al., Protein Enoineering 3(6):547-553 (1990)). Other tag polypeptides include the Flag-peptide (Hopp et al., Bio Technology, 6:1204-1210 (1988)); the KT3 epitope peptide (Martin et al., Science, 255:192-194 (1992)); an .alpha.-tubulin epitope peptide (Skinner et al., J. Biol. Chem., 266:15163-15166 (1991)); and the T7 gene 10 protein peptide tag (Lutz-Freyermuth et al., Proc. Natl. Acad. Sci. USA, 87:6393-6397 (1990)). A preferred tag is the FLAG tag.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a co-expressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant protein expression in E. coli is to express the protein in host bacteria with an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerevisiae include pYepSec1 (Baldari et al. (1987) EMBO J. 6:229-234), pMFa (Kurjan and Herskowitz, (1982) Cell 30:933-943), pJRY88 (Schultz et al. (1987) Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells, see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, by the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operably linked to a regulatory sequence in a manner that allows for expression (by transcription of the DNA molecule) of an RNA molecule that is antisense to the mRNA encoding a CNGH0011 polypeptide. Regulatory sequences operably linked to a nucleic acid cloned in the antisense orientation can be chosen which direct the continuous expression of the antisense RNA molecule in a variety of cell types; for instance, viral promoters and/or enhancers, or regulatory sequences can be chosen which direct constitutive, tissue specific, or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (Reviews—Trends in Genetics, Vol. 1(1) 1986).

Another aspect of the invention pertains to host cells into which a recombinant expression vector of the invention has been introduced. The terms “host cell” and “recombinant host cell” are used interchangeably herein. It is understood that such terms refer not only to the particular subject cell, but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells). A number of suitable mammalian host cell lines capable of expressing intact glycosylated polypeptides have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org).

Expression vectors for these cells can include one or more of the following expression control sequences: a promoter, an enhancer, and/or processing information sites, such as ribosome binding sites, RNA splice sites, polyadenylation sites, and transcriptional terminator sequences (See, e.g., Ausubel et al., supra; Sambrook, et al., supra).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those that confer resistance to drugs, such as G418, hygromycin and methotrexate. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce a CNGH0011 polypeptide. Accordingly, the invention further provides methods for producing a CNGH0011 polypeptide using the host cells of the invention. In one embodiment, the method comprises culturing the host cell of the invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can also be used to produce nonhuman transgenic animals. For example, in one embodiment, a host cell of the invention is a fertilized oocyte or an embryonic stem cell into which at least one sequence encoding a CNGH0011 polypeptide has been introduced. Such host cells can then be used to create non-human transgenic animals in which exogenous sequences encoding a CNGH0011 polypeptide have been introduced into their genome, or homologous recombinant animals in which endogenous sequences encoding a CNGH0011 polypeptide have been altered. Such animals are useful for studying the function and/or activity of the polypeptide and for identifying and/or evaluating modulators of polypeptide activity. As used herein, a “transgenic animal” is a non-human animal, preferably a mammal, more preferably a rodent, such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing nucleic acid encoding a CNGH0011 polypeptide (or a homolog thereof) into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the CNGH0011 polypeptide to particular cells. Methods for generating transgenic animals via embryo manipulation and microinjection, particularly animals such as mice, have become conventional in the art and are described, for example, in U.S. Pat. Nos. 4,736,866, 4,870,009, and 4,873,191 and in Hogan, Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

To create a homologous recombinant animal, a vector is prepared which contains at least a portion of a gene encoding a CNGH0011 polypeptide into which a deletion, addition or substitution has been introduced to thereby alter, e.g., functionally disrupt, the gene. In a preferred embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acids of the gene to allow for homologous recombination to occur between the exogenous gene carried by the vector and an endogenous gene in an embryonic stem cell. The additional flanking nucleic acid sequences are of sufficient length for successful homologous recombination with the endogenous gene. Typically, several kilobases of flanking DNA (both at the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation) and cells in which the introduced gene has homologously recombined with the endogenous gene are selected (see, e.g., Li et al. (1992) Cell 69:915). The selected cells are then injected into a blastocyst of an animal (e.g., a mouse) to form aggregation chimeras (see, e.g., Bradley in Teratocarcinomas and Embryonic Stem Cells: A Practical Approach, Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication NOS. WO 90/11354, WO 91/01140, WO 92/0968, and WO 93/04169.

In another embodiment, transgenic non-human animals can be produced which contain selected systems that allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/oxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication Nos. WO 97/07668 and WO 97/07669.

IV. Pharmaceutical Compositions

The CNGH0011 nucleic acid molecules, polypeptides, and antibodies can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In another aspect, the invention relates to CNGH0011 polypeptides or antibodies of the invention, as described herein, which are modified by the covalent attachment of a moiety. Such modification can produce a CNGH0011 polypeptide or anibody with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms. As used herein, the term “fatty acid” encompasses mono-carboxylic acids and di-carboxylic acids. Fatty acids and fatty acid esters suitable for modifying antibodies of the invention can be saturated or can contain one or more units of unsaturation. Fatty acids that are suitable for modifying antibodies of the invention include, for example, n-dodecanoate (Cl₂, laurate), n-tetradecanoate (C₁₄, myristate), n-octadecanoate (C₁₈, stearate), n-eicosanoate (C₂₀, arachidate), n-docosanoate (C₂₂, behenate), n-triacontanoate (C₃₀), n-tetracontanoate (C₄₀), cis-delta 9-octadecanoate (C₁₈, oleate), all cis-delta5,8,11,14-eicosatetraenoate (C₂₀, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably, one to about six, carbon atoms.

The modified human polypeptides and antibodies can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)).

The invention includes methods for preparing pharmaceutical compositions for modulating the expression or activity of a CNGH0011 polypeptide, nucleic acid, or antibody. Such methods comprise formulating a pharmaceutically acceptable carrier with an agent that modulates expression or activity of a CNGH0011 polypeptide, nucleic acid, or antibody. Such compositions can further include additional active agents. Thus, the invention further includes methods for preparing a pharmaceutical composition by formulating a pharmaceutically acceptable carrier with an agent that modulates expression or activity of a CNGH0011 polypeptide, nucleic acid, or antibody and one or more additional active compounds.

The agent that modulates expression or activity can, for example, be a small molecule. For example, such small molecules include peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents and protein or polypeptide agents depend upon a number of factors within the knowledge of the ordinarily skilled physician, veterinarian, or researcher. The dose(s) of these agents will vary, for example, depending upon the identity, size, and condition of the subject or sample being treated, further depending upon the route by which the composition is to be administered, if applicable, and the effect which the practitioner desires the agent to have upon the CNGH0011 polypeptide, nucleic acid, or antibody. Exemplary doses of a small molecule include milligram or microgram amounts per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). Exemplary doses of a protein or polypeptide include gram, milligram or microgram amounts per kilogram of subject or sample weight (e.g., about 1 microgram per kilogram to about 5 grams per kilogram, about 100 micrograms per kilogram to about 500 milligrams per kilogram, or about 1 milligram per kilogram to about 50 milligrams per kilogram). It is furthermore understood that appropriate doses of one of these agents depend upon the potency of the agent with respect to the expression or activity to be modulated. Such appropriate doses can be determined using the assays described herein. When one or more of these agents is to be administered to an animal (e.g., a human) in order to modulate expression or activity of a CNGH0011 polypeptide, nucleic acid, or antibody, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation or buccal), transdermal (topical), transmucosal, and rectal administration. Solutions or suspensions used for parenteral, intradermal, or subcutaneous application can include the following components: a sterile diluent, such as water for injection, saline solution, fixed oils, polyethylene glycols, glycerine, propylene glycol or other synthetic solvents; antibacterial agents, such as benzyl alcohol or methyl parabens; antioxidants, such as ascorbic acid or sodium bisulfite; chelating agents, such as ethylenediamine-tetraacetic acid; buffers, such as acetates, citrates or phosphates and agents for the adjustment of tonicity, such as sodium chloride or dextrose. The pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating, such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride, in the composition. Prolonged absorption of the injectable compositions can be brought about by including in the composition an agent which delays absorption, for example, aluminum monostearate and gelatin. Pharmaceutical excipients and additives useful in stabilizing the present composition include, but are not limited to, polypeptides, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary but non-limiting polypeptide excipients include serum albumin, such as human serum albumin (HSA), recombinant human albumin (rHA), gelatin, casein, and the like. Representative amino acids, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium, and then incorporating the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. Non-limiting examples of, and methods of preparing such sterile solutions are well known in the art, such as, but limited to, Gennaro, Ed., Remington's Pharmaceutical Sciences, 18^{th} Edition, Mack Publishing Co. (Easton, Pa.) 1990.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches, and the like can contain any of the following ingredients, or compounds of a similar nature: a binder, such as microcrystalline cellulose, gum tragacanth or gelatin; an excipient, such as starch or lactose, a disintegrating agent, such as alginic acid, Primogel, or corn starch; a lubricant, such as magnesium stearate or Sterotes; a glidant, such as colloidal silicon dioxide; a sweetening agent, such as sucrose or saccharin; or a flavoring agent, such as peppermint, methyl salicylate, or orange flavoring.

For administration by inhalation, the compounds are delivered in the form of aerosolized particles from a pressurized container or dispenser that contains a suitable propellant, e.g., a gas such as carbon dioxide, or a nebulizer. Alternatively, compositions formulated as particles can be dispersed by electrostatic, mechanical means including vibrations, or ultrasonic means as taught in U.S. Pat. Nos. 4,530,464; 4,533,082; 5,838,350; 6,113,001; 6,514,496; 5,518,179; 5,152,456; 5,261,601; and 4,605,167.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams, as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions (including liposomes having monoclonal antibodies incorporated therein or thereon) can also be used as pharmaceutically acceptable carriers. Particularly preferred compositions and methods are taught in U.S. Pat. Nos. 5,891,468 and 6,316,024.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is about 0.1 mg/kg to 100 mg/kg of body weight (generally about 10 mg/kg to 20 mg/kg). If the antibody is to act in the brain, a dosage of about 50 mg/kg to 100 mg/kg is usually appropriate. Generally, partially human antibodies and fully human antibodies have a longer half-life within the human body than other antibodies. Accordingly, the use of lower dosages and less frequent administration is often possible. Modifications, such as lipidation, can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The CNGH0011 nucleic acid molecules can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologs, and antibodies described herein can be used in one or more of the following methods: (a) screening assays; (b) detection assays (e.g., chromosomal mapping, tissue typing, forensic biology); (c) predictive medicine (e.g., diagnostic assays, prognostic assays, monitoring clinical trials, and pharmacogenomics); and (d) methods of treatment (e.g., therapeutic and prophylactic). For example, CNGH0011 polypeptides can be used for all of the purposes identified herein in portions of the disclosure relating to individual types of CNGH0011 proteins. The isolated CNGH0011 nucleic acid molecules can be used to express proteins (e.g., via a recombinant expression vector in a host cell in gene therapy applications), to detect mRNA (e.g., in a biological sample) or a genetic lesion, and to modulate activity of a CNGH0011 polypeptide. In addition, the CNGH0011 polypeptides can be used to screen drugs or compounds which modulate activity or expression of a CNGH0011 polypeptide as well as to treat disorders characterized by insufficient or excessive production of a CNGH0011 protein or production of a form of a CNGH0011 protein which has decreased or aberrant activity compared to the wild type protein. In addition, the antibodies of the invention can be used to detect and isolate a CNGH0011 protein or to modulate activity of a CNGH0011 protein.

CNGH0011 polypeptides may also be used to identify lead compounds for drug development. The structure of the peptides described herein can be readily determined by a number of methods, such as NMR and X-ray crystallography. A comparison of the structures of peptides similar in sequence, but differing in the biological activities that they elicit in target molecules can provide information about the structure-activity relationship of the target. Information obtained from the examination of structure-activity relationships can be used to design either modified peptides, or other small molecules or lead compounds which can be tested for predicted properties as related to the target molecule. The activity of the lead compounds can be evaluated using assays similar to those described herein.

Information about structure-activity relationships may also be obtained from co-crystallization studies. In these studies, a peptide with a desired activity is crystallized in association with a target molecule, and the X-ray structure of the complex is determined. The structure can then be compared to the structure of the target molecule in its native state, and information from such a comparison may be used to design compounds expected to possess desired activities.

The invention also contemplates methods for identifying novel compounds that bind to the CNGH0011 peptides, thereby affecting a CNGH0011-signaling pathway. Protein-protein interactions may be identified using conventional methods, such as co-immunoprecipitation, crosslinking and co-purification through gradients or chromatographic columns. Methods may also be employed that result in the simultaneous identification of genes which encode proteins interacting with a molecule. These methods include probing expression libraries with labeled molecules. Additionally, x-ray crystallographic studies may be used as a means of evaluating interactions with substances and molecules.

Mature CNGH0011 or its analogs or ligand can be used to modulate, i.e., increase or decrease, cytokine, chemokine and other growth factor production in response to intrinsic or extrinsic stimulation. Since CNGH0011 can transduce extracellular signals and elicit cellular responses, mature CNGH0011 or its analogs or ligand(s) can also be used to treat various kinds of immune-mediated inflammatory diseases that are dependent on inflammatory cytokines and chemokines, such as asthma, COPD, and emphysema. In summary, mature CNGH0011 or its analogs can be used alone or in combination with an antigen as an adjuvant to treat or prevent various immune mediated inflammatory diseases.

This invention further pertains to novel agents identified by the above-described screening assays and uses thereof for treatments as described herein.

A. Screening Assays

The invention provides a method (also referred to herein as a “screening assay”) for identifying modulators, i.e., candidate or test compounds or agents (e.g., peptides, peptidomimetics, small molecules or other drugs) which bind to a CNGH0011 polypeptide or have a stimulatory or inhibitory effect on, for example, expression or activity of a CNGH0011 polypeptide.

In one embodiment, the invention provides assays for screening candidate or test compounds that bind to or modulate the activity of the membrane-bound form of a CNGH0011 polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds can be presented in solution (e.g., Houghten (1992) Bio/Techniques 13:412-421), or on beads (Lam (1991) Nature 354:82-84), glass slides also known as “gene chips” (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

In one embodiment, an assay is a cell-based assay in which a cell that expresses a membrane-bound form of a CNGH0011 polypeptide, or a biologically active portion thereof, on the cell surface is contacted with a test compound and the ability of the test compound to bind to the polypeptide determined. The cell, for example, can be a yeast cell or a cell of mammalian origin. Determining the ability of the test compound to bind to the polypeptide can be accomplished, for example, by coupling the test compound with a radioisotope or enzymatic label such that binding of the test compound to the polypeptide or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radio-emission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In a preferred embodiment, the assay comprises contacting a cell which expresses a membrane-bound form of a CNGH0011 polypeptide, or a biologically active portion thereof, on the cell surface with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind to the polypeptide or a biologically active portion thereof as compared to the known compound.

In another embodiment, the assay involves assessment of an activity characteristic of the polypeptide, wherein binding of the test compound with the polypeptide or a biologically active portion thereof alters (i.e., increases or decreases) the activity of the polypeptide.

In another embodiment, an assay is a cell-based assay comprising contacting a cell expressing a membrane-bound form of a CNGH0011 polypeptide, or a biologically active portion thereof, on the cell surface with a test compound and determining the ability of the test compound to modulate (e.g., stimulate or inhibit) the activity of the polypeptide or biologically active portion thereof. Determining the ability of the test compound to modulate the activity of the polypeptide or a biologically active portion thereof can be accomplished, for example, by determining the ability of the polypeptide to bind to or interact with a target molecule or to transport molecules across the cytoplasmic membrane.

Determining the ability of a CNGH0011 polypeptide to bind to or interact with a target molecule can be accomplished by one of the methods described above for determining direct binding. As used herein, a “target molecule” is a molecule with which a selected polypeptide (e.g., a CNGH0011 polypeptide) binds or interacts with in nature, for example, a molecule on the surface of a cell which expresses the selected protein, a molecule on the surface of a second cell, a molecule in the extracellular milieu, a molecule associated with the internal surface of a cell membrane or a cytoplasmic molecule. A target molecule can be a CNGH0011 polypeptide or some other polypeptide or protein. For example, a target molecule can be a component of a signal transduction pathway which facilitates transduction of an extracellular signal (e.g., a signal generated by binding of a compound to a polypeptide of the invention) through the cell membrane and into the cell or a second intracellular protein which has catalytic activity or a protein which facilitates the association of downstream signaling molecules with a polypeptide of the invention. Determining the ability of a CNGH0011 polypeptide to bind to or interact with a target molecule can be accomplished by determining the activity of the target molecule. For example, the activity of the target molecule can be determined by detecting induction of a cellular second messenger of the target (e.g., an mRNA, intracellular Ca²⁺, diacylglycerol, IP3, and the like), detecting catalytic/enzymatic activity of the target on an appropriate substrate, detecting the induction of a reporter gene (e.g., a regulatory element that is responsive to a polypeptide of the invention operably linked to a nucleic acid encoding a detectable marker, e.g. luciferase), or detecting a cellular response, for example, cellular differentiation, or cell proliferation.

In yet another embodiment, an assay of the present invention is a cell-free assay comprising contacting a CNGH0011 polypeptide or biologically active portion thereof with a test compound and determining the ability of the test compound to bind to the polypeptide or biologically active portion thereof. Binding of the test compound to the polypeptide can be determined either directly or indirectly as described above. In a preferred embodiment, the assay includes contacting the CNGH0011 polypeptide or biologically active portion thereof with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind to the polypeptide or biologically active portion thereof as compared to the known compound.

In another embodiment, an assay is a cell-free assay comprising contacting a CNGH0011 polypeptide or biologically active portion thereof with a test compound and determining the ability of the test compound to modulate (e.g., stimulate or inhibit) the activity of the polypeptide or biologically active portion thereof. Determining the ability of the test compound to modulate the activity of the polypeptide can be accomplished, for example, by determining the ability of the polypeptide to bind to a target molecule by one of the methods described above for determining direct binding. In an alternative embodiment, determining the ability of the test compound to modulate the activity of the polypeptide can be accomplished by determining the ability of the polypeptide of the invention to further modulate the target molecule. For example, the catalytic activity, the enzymatic activity, or both, of the target molecule on an appropriate substrate can be determined as previously described.

In yet another embodiment, the cell-free assay comprises contacting a CNGH0011 polypeptide or biologically active portion thereof with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the polypeptide to preferentially bind to or modulate the activity of a target molecule.

The cell-free assays of the present invention are amenable to use of both a soluble form or the membrane-bound form of a CNGH0011 polypeptide. In the case of cell-free assays comprising the membrane-bound form of the polypeptide, it can be desirable to utilize a solubilizing agent such that the membrane-bound form of the polypeptide is maintained in solution. Examples of such solubilizing agents include non-ionic detergents, such as n-octylglucoside, n-dodecylglucoside, n-octylmaltoside, octanoyl-N-methylglucamide, decanoyl-N-methylglucamide, Triton X-100, Triton X-114, Thesit, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl-N,N-dimethyl-3-ammonio-1-propane sulfonate.

In one or more embodiments of the above assay methods of the present invention, it can be desirable to immobilize either the CNGH0011 polypeptide or its target molecule to facilitate separation of complexed from non-complexed forms of one or both of the proteins, as well as to accommodate automation of the assay. Binding of a test compound to the polypeptide, or interaction of the polypeptide with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-5-transferase fusion proteins or glutathione-5-transferase fusion proteins can be adsorbed onto glutathione Sepharose beads (Sigma Chemical; St. Louis, Mo.) or glutathione derivatized microtiter plates, which are then combined with the test compound or the test compound and either the non-adsorbed target protein or a polypeptide of the invention, and the mixture incubated under conditions conducive to complex formation (e.g., at physiological conditions for salt and pH). Following incubation, the beads or microtiter plate wells are washed to remove any unbound components and complex formation is measured either directly or indirectly, for example, as described above. Alternatively, the complexes can be dissociated from the matrix, and the level of binding or activity of the polypeptide of the invention can be determined using standard techniques.

Other techniques for immobilizing proteins on matrices can also be used in the screening assays of the invention. For example, either the CNGH0011 polypeptide or its target molecule can be immobilized utilizing conjugation of biotin and streptavidin. Biotinylated polypeptide of the invention or target molecules can be prepared from biotin-NHS(N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with the CNGH0011 polypeptide or target molecules but which do not interfere with binding of the CNGH0011 polypeptide to its target molecule can be derivatized to the wells of the plate, and unbound target or polypeptide of the invention trapped in the wells by antibody conjugation. Methods for detecting such complexes, in addition to those described above for the GST-immobilized complexes, include immunodetection of complexes using antibodies reactive with the CNGH0011 polypeptide or target molecule, as well as enzyme-linked assays which rely on detecting an enzymatic activity associated with the CNGH0011 polypeptide or target molecule.

In another embodiment, modulators of expression of a CNGH0011 polypeptide are identified in a method in which a cell is contacted with a candidate compound and the expression of the selected mRNA or protein (i.e., the mRNA or protein corresponding to a CNGH0011 polypeptide or nucleic acid) in the cell is determined. The level of expression of the selected mRNA or protein in the presence of the candidate compound is compared to the level of expression of the selected mRNA or protein in the absence of the candidate compound. The candidate compound can then be identified as a modulator of expression of the CNGH0011 polypeptide based on this comparison. For example, when expression of the selected mRNA or protein is greater (i.e., statistically significantly greater) in the presence of the candidate compound than in its absence, the candidate compound is identified as a stimulator of the selected mRNA or protein expression. Alternatively; when expression of the selected mRNA or protein is less (i.e., statistically significantly less) in the presence of the candidate compound than in its absence, the candidate compound is identified as an inhibitor of the selected mRNA or protein expression. The level of the selected mRNA or protein expression in the cells can be determined by methods described herein.

In yet another aspect of the invention, a CNGH0011 polypeptide can be used as a “bait protein” in a two-hybrid assay or three hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind to or interact with the CNGH0011 polypeptide and modulate activity of the CNGH0011 polypeptide. Such binding proteins are also likely to be involved in the propagation of signals by the CNGH0011 polypeptide as, for example, upstream or downstream elements of a signaling pathway involving the CNGH0011 polypeptide.

This invention further pertains to novel agents identified by the above-described screening assays and uses thereof for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. Accordingly, nucleic acid molecules described herein or fragments thereof, can be used to map the location of the corresponding genes on a chromosome. The mapping of the sequences to chromosomes is an important first step in correlating these sequences with genes associated with disease.

Briefly, genes can be mapped to chromosomes by preparing PCR primers (preferably 15-25 bp in length) from the sequence of a gene of the invention. Computer analysis of the sequence of a gene of the invention can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the gene sequences will yield an amplified fragment. For a review of this technique, see D'Eustachio et al. ((1983) Science 220:919-924).

PCR mapping of somatic cell hybrids is a rapid procedure for assigning a particular sequence to a particular chromosome. Three or more sequences can be assigned per day using a single thermal cycler. Using the CNGH0011 nucleic acid sequences to design oligonucleotide primers, sub-localization can be achieved with panels of fragments from specific chromosomes. Other mapping strategies which can similarly be used to map a gene to its chromosome include in situ hybridization (described in Fan et al. (1990) Proc. Natl. Acad. Sci. USA 87:6223-27), pre-screening with labeled flow-sorted chromosomes, and pre-selection by hybridization to chromosome specific cDNA libraries. Fluorescence in situ hybridization (FISH) of a DNA sequence to a metaphase chromosomal spread can further be used to provide a precise chromosomal location in one step. For a review of this technique, see Verma et al. (Human Chromosomes: A Manual of Basic Techniques (Pergamon Press, New York, 1988)).

Reagents for chromosome mapping can be used individually to mark a single chromosome or a single site on that chromosome, or panels of reagents can be used for marking multiple sites and/or multiple chromosomes. Reagents corresponding to non-coding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland et al. (1987) Nature 325:783-787.

Moreover, differences in the DNA sequences between individuals affected and unaffected with a disease associated with a gene of the invention can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The CNGH0011 nucleic acid sequences can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The CNGH0011 sequences are useful as additional DNA markers for RFLP (described in U.S. Pat. No. 5,272,057).

Furthermore, the CNGH0011 nucleic acid sequences can be used to provide an alternative technique that determines the actual base-by-base DNA sequence of selected portions of an individual's genome. Thus, the nucleic acid sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The CNGH0011 nucleic acid sequences of the present invention can be used to obtain such identification sequences from individuals and from tissue. The CNGH0011 nucleic acid sequences uniquely represent portions of the human genome. Allelic variation occurs to some degree in the coding regions of these sequences, and to a greater degree in the non-coding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the non-coding regions, fewer sequences are necessary to differentiate individuals. The non-coding sequences of SEQ ID NO: 1 can comfortably provide positive individual identification with a panel of perhaps 10 to 1,000 primers which each yield a non-coding amplified sequence of 100 bases. If a predicted coding sequence, such as that in SEQ ID NO: 1 from position 433 to 1482 is used, a more appropriate number of primers for positive individual identification would be 500-2,000.

If a panel of reagents from the nucleic acid sequences described herein is used to generate a unique identification database for an individual, those same reagents can later be used to identify tissue from that individual. Using the unique identification database, positive identification of the individual, living or dead, can be made from extremely small tissue samples.

3. Use of Partial Gene Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen, found at a crime scene. The amplified sequence can then be compared to a standard, thereby allowing identification of the origin of the biological sample.

The CNGH0011 nucleic acid sequences can be used to provide polynucleotide reagents, e.g., PCR primers, targeted to specific loci in the human genome, which can enhance the reliability of DNA-based forensic identifications by, for example, providing another “identification marker” (i.e., another DNA sequence that is unique to a particular individual). As mentioned above, actual base sequence information can be used for identification as an accurate alternative to patterns formed by restriction enzyme generated fragments. Sequences targeted to non-coding regions are particularly appropriate for this use as greater numbers of polymorphisms occur in the non-coding regions, making it easier to differentiate individuals using this technique. Examples of polynucleotide reagents include the CNGH0011 nucleic acid sequences or portions thereof, e.g., fragments derived from non-coding regions having a length of at least 20 or 30 bases.

The nucleic acid sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such probes can be used to identify tissue by species and/or by organ type.

C. Predictive Medicine

The present invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining expression of a CNGH0011 polypeptide or nucleic acid and/or activity of a CNGH0011 polypeptide, in the context of a biological sample (e.g., blood, serum, cells, tissue) to thereby determine whether an individual is afflicted with a disease or disorder, or is at risk of developing a disorder, associated with aberrant expression or activity of a CNGH0011 polypeptide. The invention also provides for prognostic (or predictive) assays for determining whether an individual is at risk of developing a disorder associated with aberrant expression or activity of a CNGH0011 polypeptide. For example, mutations in a CNGH0011 gene can be assayed in a biological sample. Such assays can be used for prognostic or predictive purpose to thereby prophylactically treat an individual prior to the onset of a disorder characterized by or associated with aberrant expression or activity of a CNGH0011 polypeptide.

Another aspect of the invention provides methods for expression of a CNGH0011 nucleic acid or polypeptide or activity of a CNGH0011 polypeptide in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent).

Yet another aspect of the invention pertains to monitoring the influence of agents (e.g., drugs or other compounds) on the expression or activity of a CNGH0011 polypeptide in clinical trials. These and other agents are described in further detail in the following sections.

1. Diagnostic Assays

An exemplary method for detecting the presence or absence of a CNGH0011 polypeptide or nucleic acid in a biological sample involves obtaining a biological sample from a test subject and contacting the biological sample with a compound or an agent capable of detecting a CNGH0011 polypeptide or nucleic acid (e.g., mRNA, genomic DNA) such that the presence of a CNGH0011 polypeptide or nucleic acid is detected in the biological sample. A preferred agent for detecting mRNA or genomic DNA encoding a CNGH0011 polypeptide is a labeled nucleic acid probe capable of hybridizing to mRNA or genomic DNA encoding a CNGH0011 polypeptide. The nucleic acid probe can be, for example, a full-length cDNA, such as the nucleic acid of SEQ ID NO: 1 or a portion thereof, such as an oligonucleotide of at least 15, 30, 50, 100, 250 or 500 nucleotides in length and sufficient to specifically hybridize under stringent conditions to a mRNA or genomic DNA encoding a CNGH0011 polypeptide. Other suitable probes for use in the diagnostic assays of the invention are described herein.

A preferred agent for detecting a CNGH0011 polypeptide is an antibody capable of binding to a CNGH0011 polypeptide, preferably an antibody with a detectable label. Antibodies can be polyclonal or, more preferably, monoclonal. An intact antibody, or a fragment thereof (e.g., Fab or F(ab′)2) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect mRNA, protein, or genomic DNA in a biological sample in vitro as well as in vivo. For example, in vitro techniques for detection of mRNA include Northern hybridizations and in situ hybridizations. In vitro techniques for detection of a CNGH0011 polypeptide include enzyme linked immunosorbent assays (ELISAs), Western blots, immunoprecipitations and immunofluorescence. In vitro techniques for detection of genomic DNA include Southern hybridizations. Furthermore, in vivo techniques for detection of a CNGH0011 polypeptide include introducing into a subject a labeled antibody directed against the polypeptide. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting a CNGH0011 polypeptide or mRNA or genomic DNA encoding a CNGH0011 polypeptide, such that the presence of the polypeptide or mRNA or genomic DNA encoding the polypeptide is detected in the biological sample, and comparing the presence of the polypeptide or mRNA or genomic DNA encoding the polypeptide in the control sample with the presence of the polypeptide or mRNA or genomic DNA encoding the polypeptide in the test sample.

The invention also encompasses kits for detecting the presence of a CNGH0011 polypeptide or nucleic acid in a biological sample (a test sample). Such kits can be used to determine if a subject is suffering from or is at increased risk of developing a disorder associated with aberrant expression of a CNGH0011 polypeptide (e.g., one of the disorders described in the section of this disclosure wherein the individual polypeptide of the invention is discussed). For example, the kit can comprise a labeled compound or agent capable of detecting the polypeptide or mRNA encoding the polypeptide in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for observing that the tested subject is suffering from or is at risk of developing a disorder associated with aberrant expression of the polypeptide if the amount of the polypeptide or mRNA encoding the polypeptide is above or below a normal level.

For antibody-based kits, the kit can comprise, for example: (1) a first antibody (e.g., attached to a solid support) which binds to a CNGH0011 polypeptide; and, optionally, (2) a second, different antibody which binds to either the polypeptide or the first antibody and is conjugated to a detectable agent.

For oligonucleotide-based kits, the kit can comprise, for example: (1) an oligonucleotide, e.g., a detectably labeled oligonucleotide, which hybridizes to a nucleic acid sequence encoding a CNGH0011 polypeptide or (2) a pair of primers useful for amplifying a nucleic acid molecule encoding a CNGH0011 polypeptide. The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples that can be assayed and compared to the test sample contained. Each component of the kit is usually enclosed within an individual container and all of the various containers are within a single package along with instructions for observing whether the tested subject is suffering from or is at risk of developing a disorder associated with aberrant expression of the polypeptide.

2. Prognostic Assays

The methods described herein can furthermore be utilized as diagnostic or prognostic assays to identify subjects having or at risk of developing a disease or disorder associated with aberrant expression or activity of a CNGH0011 polypeptide. For example, the assays described herein, such as the preceding diagnostic assays or the following assays, can be utilized to identify a subject having or at risk of developing a disorder associated with aberrant expression or activity of a CNGH0011 polypeptide (e.g., one of the disorders described in the section of this disclosure wherein the individual CNGH0011 polypeptide is discussed). Alternatively, the prognostic assays can be utilized to identify a subject having or at risk for developing such a disease or disorder. Thus, the present invention provides a method in which a test sample is obtained from a subject and a CNGH0011 polypeptide or nucleic acid (e.g., mRNA, genomic DNA) is detected, wherein the presence of the polypeptide or nucleic acid is diagnostic for a subject having or at risk of developing a disease or disorder associated with aberrant expression or activity of the polypeptide. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant expression or activity of a CNGH0011 polypeptide. For example, such methods can be used to determine whether a subject can be effectively treated with a specific agent or class of agents (e.g., agents of a type which decrease activity of the polypeptide). Thus, the present invention provides methods for determining whether a subject can be effectively treated with an agent for a disorder associated with aberrant expression or activity of a CNGH0011 polypeptide in which a test sample is obtained and the polypeptide or nucleic acid encoding the polypeptide is detected (e.g., wherein the presence of the polypeptide or nucleic acid is diagnostic for a subject that can be administered the agent to treat a disorder associated with aberrant expression or activity of the polypeptide).

The methods of the invention can also be used to detect genetic lesions or mutations in a gene of the invention, thereby determining if a subject with the lesioned gene is at risk for a disorder characterized by aberrant expression or activity of a CNGH0011 polypeptide. In preferred embodiments, the methods include detecting, in a sample of cells from the subject, the presence or absence of a genetic lesion or mutation characterized by at least one of an alteration affecting the integrity of a gene encoding the polypeptide of the invention, or the mis-expression of the gene encoding the CNGH0011 polypeptide. For example, such genetic lesions or mutations can be detected by ascertaining the existence of at least one of: (1) a deletion of one or more nucleotides from the gene; (2) an addition of one or more nucleotides to the gene; (3) a substitution of one or more nucleotides of the gene; (4) a chromosomal rearrangement of the gene; (5) an alteration in the level of a messenger RNA transcript of the gene; (6) an aberrant modification of the gene, such as the methylation pattern of the genomic DNA; (7) the presence of a non-wild type splicing pattern of a messenger RNA transcript of the gene; (8) a non-wild type level of the protein encoded by the gene; (9) an allelic loss of the gene; and (10) an inappropriate post-translational modification of the protein encoded by the gene. As described herein, there are a large number of assay techniques known in the art that can be used for detecting lesions in a gene.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a gene (see, e.g., Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to the selected gene under conditions such that hybridization and amplification of the gene (if present) occurs, and detecting the presence or absence of an amplification product, or detecting the size of the amplification product and comparing the length to a control sample. PCR and/or LCR can be desirable to use as a preliminary amplification step in conjunction with any of the techniques used for detecting mutations described herein.

Alternative amplification methods include: self-sustained sequence replication (Guatelli et al. (1990) Proc. Natl. Acad. Sci. USA 87:1874-1878), transcriptional amplification system (Kwoh, et al. (1989) Proc. Natl. Acad. Sci. USA 86:1173-1177), Q-Beta Replicase (Lizardi et al. (1988) BioTechnology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a selected gene from a sample cell can be identified by alterations in restriction enzyme cleavage patterns. For example, sample and control DNA is isolated, (optionally) amplified, digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). For example, genetic mutations can be identified in two-dimensional arrays containing light-generated DNA probes as described in Cronin et al., supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the selected gene and detect mutations by comparing the sequence of the sample nucleic acids with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Bio/Techniques 19:448), including sequencing by mass spectrometry (see, e.g., PCT Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a selected gene include methods in which protection from cleavage agents is used to detect mismatched bases in RNA/RNA or RNA/DNA heteroduplexes (Myers et al. (1985) Science 230:1242). In general, the technique of mismatch cleavage entails providing heteroduplexes formed by hybridizing (labeled) RNA or DNA containing the wild-type sequence with potentially mutant RNA or DNA obtained from a tissue sample. The double-stranded duplexes are treated with an agent that cleaves single-stranded regions of the duplex, such as which will exist due to base pair mismatches between the control and sample strands. RNA/DNA duplexes can be treated with RNASE to digest mismatched regions, and DNA/DNA hybrids can be treated with S1 nuclease to digest mismatched regions.

In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called DNA mismatch repair enzymes) in defined systems for detecting and mapping point mutations in cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a selected sequence, e.g., a wild-type sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in genes. For example, single strand conformation polymorphism (SSCP) can be used to detect differences in electrophoretic mobility between mutant and wild type nucleic acids (Orita et al. (1989) Proc. Natl. Acad. Sci. USA 86:2766; see also Cotton (1993) Mutat. Res. 285:125-144; Hayashi (1992) Genet. Anal. Tech. Appl. 9:73-79). Single-stranded DNA fragments of sample and control nucleic acids will be denatured and allowed to renature. The secondary structure of single-stranded nucleic acids varies according to sequence, and the resulting alteration in electrophoretic mobility enables the detection of even a single base change. The DNA fragments can be labeled or detected with labeled probes. The sensitivity of the assay can be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example, by adding a ‘GC clamp’ of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, and selective primer extension. For example, oligonucleotide primers can be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification can be used in conjunction with the instant invention. Oligonucleotides used as primers for specific amplification can carry the mutation of interest in the center of the molecule (so that amplification depends on differential hybridization; Gibbs et al. (1989) Nucleic Acids Res. 17:2437-2448) or at the extreme 3′ end of one primer where, under appropriate conditions, mismatching can prevent or reduce polymerase extension (Prossner (1993) Tibtech 11:238). In addition, it can be desirable to introduce a novel restriction site in the region of the mutation to create cleavage-based detection (Gasparini et al. (1992) Mol. Cell Probes 6:1). Amplification can also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein can be performed, for example, using pre-packaged diagnostic kits comprising at least one probe nucleic acid or antibody reagent described herein, which can be conveniently used, e.g., in clinical settings to diagnose patients exhibiting symptoms or family history of a disease or illness involving a gene encoding a polypeptide of the invention. Furthermore, any cell type or tissue, preferably peripheral blood leukocytes, in which the CNGH0011 polypeptide is expressed can be utilized in the prognostic assays described herein.

3. Pharmacogenomics

Agents, or modulators that have a stimulatory or inhibitory effect on activity or expression of a CNGH0011 polypeptide as identified by a screening assay described herein, can be administered to individuals to treat (prophylactically or therapeutically) disorders associated with aberrant activity of the polypeptide. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of a CNGH0011 polypeptide, expression of a nucleic acid of the invention, or mutation content of a gene of the invention in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism.” These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of a CNGH0011 polypeptide, expression of a nucleic acid encoding the polypeptide, or mutation content of a gene encoding the polypeptide in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of activity or expression of the polypeptide, such as a modulator identified by one of the exemplary screening assays described herein.

4. Monitoring of Effects During Clinical Trials

Monitoring the influence of agents (e.g., drug compounds) on the expression or activity of a CNGH0011 polypeptide (e.g., the ability to modulate aberrant cell proliferation chemotaxis, and/or differentiation) can be applied not only in basic drug screening, but also in clinical trials. For example, the effectiveness of an agent, as determined by a screening assay as described herein, to increase gene expression, protein levels, or protein activity, can be monitored in clinical trials of subjects exhibiting decreased gene expression, protein levels, or protein activity. Alternatively, the effectiveness of an agent, as determined by a screening assay, to decrease gene expression, protein levels or protein activity, can be monitored in clinical trials of subjects exhibiting increased gene expression, protein levels, or protein activity. In such clinical trials, expression or activity of a CNGH0011 polypeptide and, preferably, that of other polypeptide that have been implicated in, for example, a cellular proliferation disorder, can be used as a marker of the immune responsiveness of a particular cell.

For example, and not by way of limitation, genes, including those of the invention, that are modulated in cells by treatment with an agent (e.g., compound, drug or small molecule) that modulates activity or expression of a CNGH0011 polypeptide (e.g., as identified in a screening assay described herein) can be identified. Thus, to study the effect of agents on cellular proliferation disorders, for example, in a clinical trial, cells can be isolated and RNA prepared and analyzed for the levels of expression of a gene of the invention and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of a gene of the invention or other genes. In this way, the gene expression pattern can serve as a marker, indicative of the physiological response of the cells to the agent. Accordingly, this response state can be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of the CNGH0011 polypeptide or nucleic acid in the pre-administration sample; (iii) obtaining one or more post-administration samples from the subject; (iv) detecting the level of the CNGH0011 polypeptide or nucleic acid in the post-administration samples; (v) comparing the level of the CNGH0011 polypeptide or nucleic acid in the pre-administration sample with the level of the CNGH0011 polypeptide or nucleic acid in the post-administration sample or samples; and (vi) altering the administration of the agent to the subject accordingly. For example, increased administration of the agent can be desirable to increase the expression or activity of the polypeptide to higher levels than detected, i.e., to increase the effectiveness of the agent. Alternatively, decreased administration of the agent can be desirable to decrease expression or activity of the polypeptide to lower levels than detected, i.e., to decrease the effectiveness of the agent.

C. Methods of Treatment

The present invention provides for both prophylactic and therapeutic methods of treating a subject at risk of (or susceptible to) a disorder or having a disorder associated with aberrant expression or activity of a CNGH0011 polypeptide and/or in which the CNGH0011 polypeptide is involved.

The present invention provides a method for modulating or treating at least one CNGH0011 related disease or condition, in a cell, tissue, organ, animal, or patient, as known in the art or as described herein, using at least one CNGH0011 peptide or CNGH0011 antibody.

Compositions of CNGH0011 binding peptides or CNGH0011 antibody or polypeptide antagonists may find therapeutic use in the treatment of disease conditions, such as cancer or other human diseases with deregulated matrix production and remodeling, including lung fibrosis, liver cirrhosis, osteoporosis, rheumatoid arthritis, and asthma. Potential disease indications may also include diseases with defects in cell mechanics, tissure structure, or deregulation of mechanochemical conversion caused by pathological alteration of matrix (Ingber D., Mechanobiology and diseases of mechanotransduction, Annals of Medicine, in press).

The present invention also provides a method for modulating or treating at least one immune related disease, in a cell, tissue, organ, animal, or patient including, but not limited to, at least one of rheumatoid arthritis, juvenile rheumatoid arthritis, systemic onset juvenile rheumatoid arthritis, psoriatic arthritis, ankylosing spondilitis, gastric ulcer, seronegative arthropathies, osteoarthritis, inflammatory bowel disease, ulcerative colitis, systemic lupus erythematosis, antiphospholipid syndrome, iridocyclitis/uveitis/optic neuritis, idiopathic pulmonary fibrosis, systemic vasculitis/wegener's granulomatosis, sarcoidosis, orchitis/vasectomy reversal procedures, allergic/atopic diseases, asthma, allergic rhinitis, eczema, allergic contact dermatitis, allergic conjunctivitis, hypersensitivity pneumonitis, transplants, organ transplant rejection, graft-versus-host disease, systemic inflammatory response syndrome, sepsis syndrome, gram positive sepsis, gram negative sepsis, culture negative sepsis, fungal sepsis, neutropenic fever, urosepsis, meningococcemia, trauma/hemorrhage, burns, ionizing radiation exposure, acute pancreatitis, adult respiratory distress syndrome, rheumatoid arthritis, alcohol-induced hepatitis, chronic inflammatory pathologies, sarcoidosis, Crohn's pathology, sickle cell anemia, diabetes, nephrosis, atopic diseases, hypersensitivity reactions, allergic rhinitis, hay fever, perennial rhinitis, conjunctivitis, endometriosis, asthma, urticaria, systemic anaphalaxis, dermatitis, pernicious anemia, hemolytic disease, thrombocytopenia, graft rejection of any organ or tissue, kidney transplant rejection, heart transplant rejection, liver transplant rejection, pancreas transplant rejection, lung transplant rejection, bone marrow transplant (BMT) rejection, skin allograft rejection, cartilage transplant rejection, bone graft rejection, small bowel transplant rejection, fetal thymus implant rejection, parathyroid transplant rejection, xenograft rejection of any organ or tissue, allograft rejection, anti-receptor hypersensitivity reactions, Graves disease, Raynoud's disease, type B insulin-resistant diabetes, myasthenia gravis, antibody-meditated cytotoxicity, type III hypersensitivity reactions, systemic lupus erythematosus, POEMS syndrome (polyneuropathy, organomegaly, endocrinopathy, monoclonal gammopathy, and skin changes syndrome), antiphospholipid syndrome, pemphigus, scleroderma, mixed connective tissue disease, idiopathic Addison's disease, diabetes mellitus, chronic active hepatitis, primary billiary cirrhosis, vitiligo, vasculitis, post-MI cardiotomy syndrome, type IV hypersensitivity, contact dermatitis, hypersensitivity pneumonitis, allograft rejection, granulomas due to intracellular organisms, drug sensitivity, metabolic/idiopathic, Wilson's disease, hemachromatosis, alpha-1-antitrypsin deficiency, diabetic retinopathy, Hashimoto's thyroiditis, osteoporosis, hypothalamic-pituitary-adrenal axis involution, primary biliary cirrhosis, thyroiditis, encephalomyelitis, cachexia, cystic fibrosis, neonatal chronic lung disease, chronic obstructive pulmonary disease (COPD), familial hematophagocytic lymphohistiocytosis, dermatologic conditions, psoriasis, alopecia, nephrotic syndrome, nephritis, glomerular nephritis, acute renal failure, hemodialysis, uremia, toxicity, preeclampsia, okt3 therapy, anti-cd3 therapy, cytokine therapy, chemotherapy, radiation therapy (e.g., including but not limited to, asthenia, anemia, cachexia, and the like), chronic salicylate intoxication, and the like. See, e.g., the Merck Manual, 12th-17th Editions, Merck & Company, Rahway, N.J. (1972, 1977, 1982, 1987, 1992, 1999), Pharmacotherapy Handbook, Wells et al., eds., Second Edition, Appleton and Lange, Stamford, Conn. (1998, 2000), each entirely incorporated by reference.

The present invention also provides a method for modulating or treating at least one malignant disease in a cell, tissue, organ, animal or patient, including, but not limited to, at least one of: leukemia, acute leukemia, acute lymphoblastic leukemia (ALL), B-cell, T-cell or FAB ALL, acute myeloid leukemia (AML), acute promyelocytic leukemia (APL), chromic myelocytic leukemia (CML), chronic lymphocytic leukemia (CLL), hairy cell leukemia, myelodyplastic syndrome (MDS), a lymphoma, Hodgkin's disease, a malignant lymphoma, Non-Hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, adenocarcinomas, sarcomas, malignant melanoma, hemangioma, metastatic disease, cancer related bone resorption, cancer related bone pain, and the like.

Disorders characterized by aberrant expression or activity of the CNGH0011 polypeptides are further described elsewhere in this disclosure.

1. Prophylactic Methods

In one aspect, the invention provides a method for at least substantially preventing in a subject, a disease or condition associated with an aberrant expression or activity of a CNGH0011 polypeptide, by administering to the subject an agent that modulates expression or at least one activity of the polypeptide. Subjects at risk for a disease that is caused or contributed to by aberrant expression or activity of a CNGH0011 polypeptide can be identified by, for example, any or a combination of diagnostic or prognostic assays as described herein. Administration of a prophylactic agent can occur prior to the manifestation of symptoms characteristic of the aberrancy, such that a disease or disorder is prevented or, alternatively, delayed in its progression. Depending on the type of aberrancy, for example, an agonist or antagonist agent can be used for treating the subject. The appropriate agent can be determined based on screening assays described herein.

2. Therapeutic Methods

Another aspect of the invention pertains to methods of modulating expression or activity of a CNGH0011 polypeptide for therapeutic purposes. The modulatory method of the invention involves contacting a cell with an agent that modulates one or more of the activities of the polypeptide. An agent that modulates activity can be an agent as described herein, such as a nucleic acid or a protein, a naturally-occurring cognate ligand of the polypeptide, a peptide, a peptidomimetic, or other small molecule. In one embodiment, the agent stimulates one or more of the biological activities of the polypeptide. Examples of such stimulatory agents include the active CNGH0011 polypeptide and a nucleic acid molecule encoding the CNGH0011 polypeptide that has been introduced into the cell. In another embodiment, the agent inhibits one or more of the biological activities of the CNGH0011 polypeptide. Examples of such inhibitory agents include antisense nucleic acid molecules and antibodies and other methods described herein. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a CNGH0011 polypeptide. In one embodiment, the method involves administering an agent (e.g., an agent identified by a screening assay described herein), or combination of agents that modulate (e.g., up-regulates or down-regulates) expression or activity. In another embodiment, the method involves administering a CNGH0011 polypeptide or nucleic acid molecule as therapy to compensate for reduced or aberrant expression or activity of the polypeptide.

Stimulation of activity is desirable in situations in which activity or expression is abnormally low or down-regulated and/or in which increased activity is likely to have a beneficial effect, e.g., in wound healing. Conversely, inhibition of activity is desirable in situations in which activity or expression is abnormally high or up-regulated and/or in which decreased activity is likely to have a beneficial effect.

### EXAMPLES

The following specific examples are intended to illustrate the invention and should not be construed as limiting the scope of the claims.

### Example 1

### CNGH0011 Sequence Data

The CNGH0011 polynucleotide of SEQ ID NO: 1 is predicted to be a 2132 base cDNA and to have no introns based on modeling with several gene prediction algorithms, such as GeneScan and Fgenesh (http://genome.ucsc.edu/) and the Acembly program (http://www.acedb.org/Cornell/acembly/) against 77 overlapping ESTs. The gene corresponding to the polynucleotide of SEQ ID NO: 1 spans nucleotides 121,073,999 to 121,076,129 on the minus strand of chromosome 2. The protein coding sequence is from nucleotides 433 to 1482 of the 2132 base cDNA.

Several previously published cDNAs share 100% sequence identity with the polynucleotide of SEQ ID NO: 1. These include a Homo sapiens cDNA with accession numbers FLJ20979 fis, ADSU01938, AK024632, Homo sapiens family with sequence similarity to 11 member B (FAM11B) retropseudogene mRNA with accession number AF530474, and a Homo sapiens cDNA encoding a protein named Family with sequence similarity 11 member B (FAM11B) with accession numbers MGC:16829, IMAGE:3850884, and BC016849. All of these nucleic acid sequences appear to encode proteins having the same amino acid sequence as SEQ ID NO:2.

### Example 2

### Predicted CNGH0011 Protein Domains

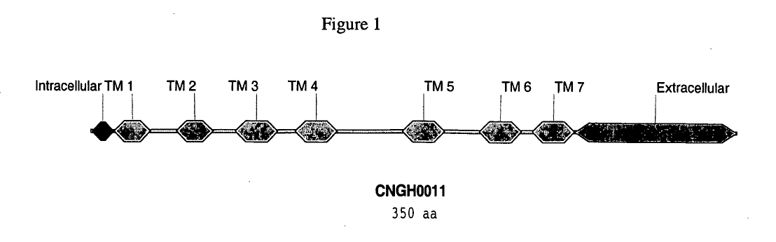

CNGH0011 is conceptually translated into a protein of 350 amino acid residues (SEQ ID NO: 2). CNGH0011 is predicted to be a 7-transmembrane (TM) protein (Table 1) (FIG. 1), a family of proteins involved in secretory pathways, by software programs such as TMHMM (E. L.L. Sonnhammer, G. von Heijne, and A. Krogh. Proceedings of the Sixth International Conference on Intelligent Systems for Molecular Biology, pages 175-182, Menlo Park, Calif., 1998. AAAI Press). As shown in Table 1 below, the topology, i.e., the portions or domains that are intracellular (in the cytoplasm or “Inside”), transmembrane (“TM”), or extracellular (“Outside”), of the CNGH0011 protein is the reverse of most G-protein coupled receptors.

TABLE 1
CNGH0011 topology predicted by TMHMM.

| Topology | Start | End |
|---|---|---|
| Inside | 1 | 12 |
| TM | 13 | 32 |
| Outside | 33 | 46 |
| TM | 47 | 66 |
| Inside | 67 | 78 |
| TM | 79 | 101 |
| Outside | 102 | 110 |
| TM | 111 | 133 |
| Inside | 134 | 168 |
| TM | 169 | 191 |
| Outside | 192 | 210 |
| TM | 211 | 233 |
| Inside | 234 | 239 |
| TM | 240 | 262 |
| Outside | 263 | 350 |

CNGH0011 is 89% identical at the amino acid sequence level to another Homo sapiens protein called Family with sequence similarity 11 member A (FAM11A) with accession number GI:20984097. FAM11A was reported to be associated with the Fragile X Syndrome linked site F (FRAXF) CpG island and to be transcriptionally silent in normal individuals carrying the FRAXF full mutation (Shaw et al. (2002) Eur. J. Hum. Genet. 10(11): 767-72). FAM11A resides on chromosome X, a different chromosome location than CNGH0011. Shaw et al. also described the FAM11B gene whose sequence is identical to SEQ ID NO: 1 (Shaw et al. supra).

Sequence homology searches using the BLASTP algorithm also revealed closely related proteins in Mus musculus and Rattus norvegicus which were designated CNGM0011 (Accession: GI:22122617; SEQ ID NO: 3) and CNGR0011 (Accession: GI:27675846; SEQ ID NO: 4), respectively. These predicted protein sequences are both 96% identical to the amino acid sequence of SEQ ID NO: 2 (Table 3). A homolog was also found in Brachydanio rerio (Zebra fish) which was designated CNGZ0011 (Accession: GI:32766321; SEQ ID NO: 5) and a homolog was found in Drosophila melanogaster which was designated CNGD0011 (Accession: GI:24643190; SEQ ID NO: 6). CNGM0011, CNGR0011, CNGZ0011, and CNGD0011 are all orthologs to CNGH0011.

TABLE 2
Percentage Identity among CNGH0011 homologs.

| | CNGH0011 | CNGM0011 | CNGR0011 | FAM11A | CNGZ0011 | CNGD0011 |
|---|---|---|---|---|---|---|
| CNGH0011 | 100 | 96 | 96 | 89 | 83 | 45 |
| CNGM0011 | | 100 | 99 | 88 | 82 | 45 |
| CNGR0011 | | | 100 | 89 | 83 | 45 |
| FAM11A | | | | 100 | 88 | 43 |
| CNGZ0011 | | | | | 100 | 42 |
| CNGD0011 | | | | | | 100 |

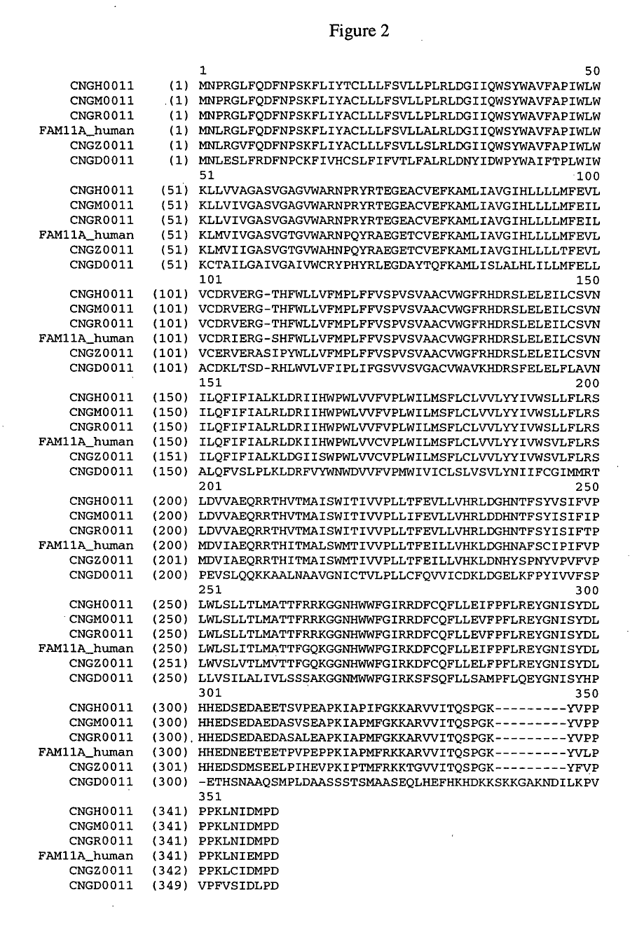

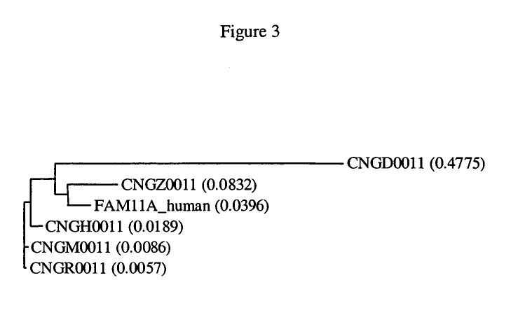

A multiple sequence alignment of the CNGH0011, CNGM0011, CNGR0011, FAM11A, CNGZ0011, and CNGD0011 proteins is provided in FIG. 2. A phylogenetic tree for these proteins is provided in FIG. 3. CNGM0011 and CNGR0011 all share a higher degree of sequence similarity, based on phylogenetic distances (FIG. 3), to CNGH0011 than FAM11A. This indicates that the CNGM0011 and CNGR0011 proteins are the true orthologs of CNGH0011. Together these CNGH0011 homologues comprise a new sub-family of the 7-transmembrane proteins. Multiple sequence alignments and phylogenetic trees were generated using Vector NTI 8.0 (InforMax, Inc., Frederick, Md.) software.

A prominent structural feature of CNGH0011 and others in this subfamily is a predicted reversed topology from a typical GPCR. In the CNGH0011 subfamily, the N-terminus before the first transmembrane domain is predicted to be intracellular and the C-terminal sequence after the last transmembrane domain is predicted to be exposed to the extracellular space. This topology is opposite to that of a typical GPCR.

The only other known 7-TM receptors with the same topology as the CNGH0011 protein are adiponectin receptor 1 and adiponectin receptor 2, which were recently discovered and designated as Type II, 7-transmembrane domain proteins. These proteins serve as receptors for globular and full-length adiponectin, and they mediate increased AMP kinase and PPAR-ligand activities, as well as fatty-acid oxidation and glucose uptake by adiponectin through G-protein-independent pathways (Yamauchi et al (2003) Nature 423:762-769). CNGH0011 and the other proteins in its sub-family belong to the type II, 7-transmembrane domain family; however, they are not related to the adiponectin receptors by sequence homology.

### Example 3

### CNGH0011 Transcipt Levels in Selected Tissues

The highest CNGH0011 mRNA transcript levels are found in seminal vesicle, bone marrow, testes, cerebellum, thymus, breast, trachea and spinal cord, although CNGH0011 is also detectable at lower levels in a variety of other tissues, such as lung, prostate, breast, liver, colon, heart, kidney, placenta, skin, pituitary, ovary, and uterus (Table 3). In human primary cells, CNGH0011 can be readily detected in lung fibroblast, umbilical artery smooth muscle cells, epidermal keratinocytes, leukocytes, and macrophage. In human tumors, CNGH0011 expression can be detected in anaplastic oligodendroglioma, nervous cell tumor, renal cell adenocarcinoma, neuroblastoma, papillary carcinoma, ductal carcinoma, adenocarcinoma, endometrial adenocarcinoma, melanotic melanoma, retinoblastoma chondrosarcoma, and parathyroid tumor.

Tissue distribution of CNGH0011 in 77 normal human tissues, primary cells, or tumors was profiled by cDNA microarray (Table 3). Total RNA from human organs was obtained from the BioChain Institute, Inc (Hayward, Calif.). RNAs were then reverse transcribed, amplified by PCR, labeled with cy5-dCTP and hybridized to microarrays containing cDNA probe sequences representing 8000 individual human genes. Fluorescence intensities were then normalized across the arrays and background fluorescence was determined using standard methods and GeneSpring 5.0 (Silicon Genetics, Redwood City, Calif.). Average fluorescence intensity was collected from three replicate arrays and corrected for background fluorescence.

TABLE 3
CNGH0011 transcipt levels in selected tissues.

| | | | Standard | | |
|---|---|---|---|---|---|
| | Average | Back- | Error of | | Exp. |
| Tissue | Intensity | ground | Mean | CloneCV | CV |
| Seminal Vesicle | 184 | 55 | 13.54 | 12.73 | 8.92 |
| Bone marrow | 181 | 26 | 1.97 | 1.88 | 6.45 |
| Testes | 146 | 27 | 5.95 | 7.05 | 7.95 |
| Cerebellum | 141 | 30 | 1.51 | 1.85 | 8.16 |
| Thymus | 138 | 23 | 7.13 | 8.91 | 11.9 |
| Breast, Female (F) | 136 | 28 | 2.74 | 3.46 | 8.96 |
| Brain total | 128 | 28 | 3.21 | 4.32 | 7.53 |
| Trachea | 122 | 26 | 1.02 | 1.45 | 7.59 |
| Spinal Cord | 117 | 26 | 4.22 | 6.2 | 13.96 |
| Small Intestine | 105 | 27 | 1.98 | 3.25 | 5.8 |
| Ovary | 105 | 19 | 3.6 | 4.83 | 10.79 |
| Lung, Male (M) | 103 | 21 | 2.94 | 4.9 | 8.83 |
| Heart ventricle | 103 | 51 | 4.79 | 8 | 13.51 |
| Frontal lobe | 102 | 39 | 3.7 | 6.23 | 13.32 |
| Abdominal adipose | 101 | 63 | 2.12 | 3.61 | 6.69 |
| Colon (F) | 99 | 19 | 12.22 | 21.33 | 14.02 |
| Spleen (F) | 98 | 19 | 2.06 | 3.64 | 9.17 |
| Gallbladder | 97 | 31 | 5.86 | 10.4 | 20.79 |
| Thalamus | 97 | 64 | 1.1 | 1.96 | 7.19 |
| Lymph node | 96 | 32 | 1.15 | 2.07 | 13.53 |
| Hippocampus | 96 | 39 | 8.06 | 14.47 | 20.27 |
| Colon descending | 95 | 44 | 5.38 | 9.72 | 7.19 |
| Colon/M | 95 | 29 | 1.36 | 2.47 | 7.33 |
| Pancreas | 95 | 54 | 4 | 7.24 | 8.26 |
| Pons | 94 | 55 | 0.8 | 1.46 | 8.86 |
| Skin (F) | 93 | 36 | 2.79 | 5.19 | 6.3 |
| Occipital lobe | 93 | 42 | 7.04 | 13.11 | 11.95 |
| Salivary Gland | 89 | 40 | 2.97 | 5.77 | 10.08 |
| Fallopian tube | 89 | 23 | 1.15 | 1.82 | 8.94 |
| Transverse colon | 89 | 37 | 4.31 | 8.32 | 10.5 |
| Bronchi | 89 | 61 | 3.97 | 7.68 | 8.05 |
| Parietal cortex | 89 | 45 | 2.94 | 5.67 | 7.17 |
| Skel muscle (F) | 88 | 32 | 6.81 | 13.36 | 7.23 |
| Cervix (F) | 87 | 21 | 7.98 | 15.7 | 7.9 |
| Heart (M) | 85 | 32 | 0.61 | 1.24 | 7.45 |
| Vulva (F) | 83 | 25 | 4.46 | 9.24 | 14.45 |
| Mid Tegmentum | 83 | 56 | 4.68 | 9.75 | 6.84 |
| Cerebellum | 82 | 45 | 1 | 2.1 | 9.64 |
| Frontal cortex | 81 | 47 | 4.01 | 8.55 | 15.82 |
| Uterus | 79 | 17 | 0.8 | 1.43 | 8.96 |
| Putamen | 79 | 46 | 2.22 | 4.81 | 12.52 |
| Kidney | 78 | 23 | 1.51 | 2.7 | 10.95 |
| Olfactory Bulb | 78 | 58 | 0.12 | 0.26 | 6.86 |
| Med Oblongata | 77 | 58 | 1.34 | 2.98 | 5.29 |
| Heart (F) | 76 | 25 | 1.61 | 3.63 | 8.33 |
| Aorta | 76 | 60 | 2.71 | 6.12 | 10.55 |
| Placenta | 75 | 18 | 2.52 | 5.79 | 10.93 |
| Substantia Nigra | 75 | 57 | 1.45 | 3.35 | 5.53 |
| Vena Cava | 75 | 56 | 2.37 | 5.41 | 7.93 |
| Bone marrow | 74 | 69 | 2.7 | 6.31 | 7.86 |
| Rectum | 73 | 16 | 1.81 | 4.25 | 10.37 |
| Vas Deferens | 73 | 58 | 0.4 | 0.95 | 7.84 |
| Kidney | 72 | 20 | 1.42 | 3.39 | 7.57 |
| Skel muscle (M) | 72 | 47 | 1.09 | 2.58 | 6.22 |
| Duodenum | 72 | 40 | 0.42 | 1.01 | 9.46 |
| Brain Dorsal Root | 72 | 55 | 1.66 | 3.95 | 12.55 |
| Temporal cortex | 72 | 46 | 0.79 | 1.88 | 6.24 |
| Hypothalamus | 71 | 49 | 2.06 | 4.98 | 6.7 |
| Bladder | 70 | 44 | 1.99 | 4.86 | 6.39 |
| Liver (F) | 69 | 28 | 7.75 | 19.3 | 13.69 |
| Liver (M) | 69 | 25 | 2.89 | 7.2 | 7.7 |
| Stomach fundus | 69 | 58 | 1.13 | 2.83 | 7.16 |
| Temporal lobe | 68 | 46 | 2.98 | 7.53 | 7.77 |
| Colon ascending | 66 | 34 | 0.74 | 1.94 | 8.4 |
| Stomach | 66 | 19 | 2.85 | 7.39 | 8.84 |
| Caudate nucleus | 66 | 41 | 6 | 15.55 | 9.39 |
| Spinal Cord | 66 | 63 | 5.19 | 13.58 | 12.67 |
| Thyroid (F) | 65 | 19 | 1.54 | 4.09 | 6.84 |
| Cecum | 65 | 38 | 3.73 | 9.93 | 7.52 |
| Stomach (F) | 64 | 35 | 1.6 | 4.26 | 6.6 |
| Sigmoid colon | 63 | 40 | 5.31 | 14.48 | 12.39 |
| Cerebellum | 62 | 54 | 0.33 | 0.91 | 8.03 |
| Colon/Human tissue | 61 | 39 | 1.49 | 4.17 | 7.7 |
| Occipital cortex | 61 | 41 | 1.64 | 4.6 | 8.79 |
| Appendix | 50 | 34 | 6.27 | 17.61 | 7.05 |

### Example 4

### Elevated Transcription of CNGH0011 in Asthmatic Tissues

As shown in Table 4 below, cDNA microarray analyses indicate that CNGH0011 gene transcript levels are elevated in asthmatic lung tissues relative to non-asthmatic control tissues.

Primary cultures of human airway smooth muscle cells (HASMC) for cDNA microarray analyses were prepared from the lung tissues of asthmatic and non-asthmatic donors (Table 4) using standard methods. HASMC were cultured in Dulbecco's Modified Eagle Medium (D-MEM) (Invitrogen, Inc., Carlsbad, Calif.) containing 10% fetal bovine serum (FBS) for up to for 4 weeks under standard conditions. HASMC were then quiesced by placing them in D-MEM containing 1% FBS for 24 h, washing the cells, and placing them in D-MEM lacking serum for 24 hours. Quiesced HASMC were then exposed to either D-MEM containing 10% atopic serum isolated from donors hypersensitive to various allergenic antigens or D-MEM containing 10% non-atopic serum isolated from normal donors. The four treatment groups were asthmatic HASMC treated with atopic sera, asthmatic HASMC treated with non-atopic sera, non-asthmatic HASMC treated with atopic sera, and non-asthmatic HASMC treated with non-atopic sera (Table 4). After serum exposure total RNA was isolated from the HASMC at time 0, 15 minutes, 30 minutes, 2 hours, 4 hours, 8 hours and 24 hours. cDNAs were then produced for each of the four treatment groups by reverse transcription, amplified by PCR, and labeled with cy5-dCTP using standard methods.

Microarrays containing cDNA probe sequences representing 8000 individual human genes were then hybridized under standard conditions (5×SSC, 0.2% SDS, 1 mg/ml Cot-1 DNA) to the labeled cDNAs, and raw fluorescence intensity data was collected from each chip using a Agilent's DNA Microarray Scanner and Feature Extraction Software (Agilent Technologies, Palo Alto, Calif.). Fluorescence intensity data was collected from at least three microarrays representing HASMC from biopsy samples falling within each of the four treatment groups. Fluorescence intensities were then normalized across the chips using standard methods and GeneSpring 5.0 (Silicon Genetics, Redwood City, Calif.). Intensity data was then analyzed by one-way ANOVA with GeneSpring 5.0 using the manufacturer's default settings and a P-value of less than 0.05. This analysis identified several genes which were up regulated at least 2 fold, relative to controls, in the HASMC prepared from asthmatic lung tissues exposed to atopic serum and HASMC from asthmatic lung tissues treated with non-atopic serum. Examination of database annotations for the genes identified in this initial screening revealed that more than 40 of the genes identified had previously been reported to be up-regulated in asthmatic tissues and confirmed the ability of the array analysis to detect such genes.

At the 8-hour time point, transcript levels of the CNGH0011 gene (SEQ ID NO: 1) were increased 5.1 fold in primary cultures of human airway smooth muscle cells (HASMC) prepared from asthmatic lung tissues exposed to atopic serum relative to identically treated non-asthmatic lung tissues. CNGH0011 transcipt levels were also increased 4.2 fold in asthmatic lung tissues treated with non-atopic serum relative to identically treated non-asthmatic lung tissues. Importantly, data collected from total RNA prepared from HASMC from the four treatment groups at the other time ponts, i.e., 15 minutes, 30 minutes, 2 hours, 4 hours, and 24 hours, indicated that the fold upregulation of CNH0011 observed at the 8-hour time point was part of a trend of increasing CNGH0011 transcript levels. This indicates a correlation between increased CNGH0011 transcript levels and the asthmatic condition.

TABLE 4
HASMC treatment groups and sources.

| HASMC | |
|---|---|
| Treatment Group | HASMC Sources |
| Asthmatic HASMC | Patient 2723, Female, 44 year-old, |
| Treated with Atopic | Asthmatic Lung Biopsy |
| Sera | Patient 2702, Female, 47 year-old, |
| Asthmatic HASMC | Asthmatic Lung Biopsy |
| Treated with | |
| Non-Atopic Sera | |
| Non-Asthmatic | Patient 2490, Female, ˜50 year-old, Lung |
| HASMC Treated with | Transplant for Hemorrhage |
| Atopic Sera | Patient 2599, Male, 50 year-old, |
| Non-Asthmatic | Lung Transplant for Emphysema |
| HASMC Treated with | Patient 2724, Male, 20 year-old, |
| Non-Atopic Sera | Non-Asthmatic Lung Biopsy |
| | Patient 2204, Female, 47 year-old, Lung |
| | Resection for Large Cell Carcinoma (CA) |

### Example 5

### Effect of LPS on CNGH0011 Transcipt Levels in Human White Blood Cells

CNGH0011 transcript levels are increased in white blood cells (WBC) isolated from human subjects receiving lipopolysaccharide (LPS) intravenously (Table 5). This data indicates that CNGH0011 transcription is increased as part of the immune response to LPS and is consistent with the increased transcription of CNGH0011 (from Example 4 above) observed in asthmatic HASMC treated with atopic sera from patients with hypersensitive immune responses.

For this study, two human subjects at Pharma Bio-Research International (Zuidlaren, Netherlands) received LPS intravenously at 4 ng/kg and blood samples were taken prior to LPS treatment and at 2 hours, 3 hours, 6 hours, 10 hours and 24 hours after LPS injection. WBC were then isolated from the samples and total RNA was extracted from these cells using standard methods. RNAs were then reverse transcribed, PCR amplified, labeled with cy5-dCTP and hybridized to microarrays containing cDNA probe sequences representing 8000 individual human genes. Average fluorescence intensity was collected from three replicate arrays. Fluorescence intensities were then normalized across the arrays and background fluorescence was determined using standard methods and GeneSpring 5.0 (Silicon Genetics, Redwood City, Calif.). Average fluorescence intensity was collected from three replicate arrays and corrected for background fluorescence.

TABLE 5
Effect of LPS on CNGH0011 transcipt levels in human white blood cells

| | | Average | | Standard | | |
|---|---|---|---|---|---|---|
| Donor | | Inten- | Back- | Error of | Clone | Exp. |
| # | Treatment | sity | ground | Mean | CV | CV |
| Donor4 | No LPS/control | 87 | 9 | 4.34 | 8.56 | 17.88 |
| Donor5 | No LPS/control | 85 | 25 | 4.2 | 8.53 | 30.42 |
| Donor4 | LPS 2 hr/treated | 212 | 17 | 22.05 | 14.7 | 19.56 |
| Donor5 | LPS 2 hr/treated | 78 | 30 | 26.45 | 47.83 | 36.81 |
| Donor4 | LPS 3 hr/treated | 383 | 16 | 14.74 | 6.66 | 18.14 |
| Donor5 | LPS 3 hr/treated | 291 | 24 | 6.36 | 3.09 | 29.64 |
| Donor4 | LPS 6 hr/treated | 211 | 12 | 5.49 | 3.68 | 22.48 |
| Donor5 | LPS 6 hr/treated | 215 | 18 | 12.51 | 10.04 | 20.18 |
| Donor4 | LPS 10 hr/treated | 161 | 18 | 13.68 | 11.95 | 17.19 |
| Donor5 | LPS 10 hr/treated | 135 | 28 | 0 | 0 | 2.19 |
| Donor4 | LPS 24 hr/treated | 86 | 16 | 0 | 0 | 2.32 |
| Donor5 | LPS 24 hr/treated | 89 | 23 | 3.25 | 5.12 | 35.14 |

### Example 6

### Silencing RNAs can Decrease CNGH0011 Transcript Levels

CNGH0011 transcript levels in primary human lung fibroblast HFL1 cells may be decreased by transient transfection with the T1 and T3 silencing RNAs (siRNA). The double stranded T1 and T3 siRNA molecules were designed to target CNGH0011 transcripts using the criteria of Reynolds et al. (see Reynolds et al., Nature Biotechnology, 22(3): 326-30 (2004)) and were confirmed by BLASTN to have at least 3 mismatches relative to any other known mRNA sequence. The T1 (SEQ ID NO: 7 and SEQ ID NO: 8) and T3 (SEQ ID NO: 9 and SEQ ID NO: 10) siRNAs each comprise a double stranded, 19 base pair RNA strand with asymmetrical deoxythymidine (dTdT) overhangs at the 3′ end of each strand.

The T1 and T3 siRNA molecules were administered by transfection to HFL1 cells grown in Ham's F12K medium supplemented with 2 mM L-glutamine, 1.5 g/L sodium bicarbonate, and 10% FBS. Cells were grow under standard conditions at 37° C. in an atmosphere containing 5% CO₂. Prior to transfection, 2×10⁵ cells/ml were seeded into each well of a six well plate and allowed to recover for 24 hours. Cells were then transfected as indicated in Table 6 using Lipofectamine™ 2000 (Invitrogen Corp., Carlsbad, Calif.). Cells were harvested by trypsinization 24 hours after transfection and a pool of total RNA from four identically transfected wells was prepared using the RNAeasy® Mini System (Qiagen Inc., Valencia, Calif.). Total RNA for each control was then assayed in triplicate by quantitative real-time PCR (Q-PCR) to determine CNGH0011 and hypoxanthine phosphoribosyltransferase (HPRT) transcript levels in multiplexed assays. Q-PCR and primer set design was performed using the BD QZyme™ Assay System (BD Biosciences, Palo Alto, Calif.) as directed by the manufacturer.

The data in Table 6 indicates that the T1 and T3 siRNAs can decrease CNGH0011 mRNA transcript levels. CNGH0011 transcript levels were normalized to HPRT transcript levels and then expressed as a percentage of the HPTRT normalized CNGH0011 transcript levels observed in mock transfected HFL1 cells. The parenthetical notations (a) and (b) represent values from two independently conducted experiments.

TABLE 6
Effect of siRNAs on CNGH0011 transcipt levels in HFL1 cells.

| | Percentage of CNGH0011 | |
|---|---|---|
| Transfection Sample | Transcript Level Relative to Mock | |
| (Experiment) | Transfected Cells | Error (±) |
| mock (a) | 100 | 23 |
| mock (b) | 100 | 16 |
| Scrambled (a) | 116 | 21 |
| Scrambled (b) | 97 | 14 |
| siRNA T1 (a) | 67 | 14 |
| siRNA T1 (b) | 64 | 13 |
| siRNA T3 (a) | 64 | 13 |
| siRNA T3 (b) | 68 | 9 |

Although illustrated and described above with reference to certain specific embodiments, the present invention is nevertheless not intended to be limited to the details shown. Rather, the present invention is directed to the CNGH0011 polypeptides, polynucleotides, antibodies, apparatus, and kits disclosed herein and uses thereof, and various modifications may be made in the details within the scope and range of equivalents of the claims and without departing from the spirit of the invention.

1. An isolated CNGH0011 polypeptide comprising the amino acid sequence of SEQ ID NO: 2.

2. An isolated CNGH0011 polypeptide comprising at least one sequence corresponding to:
an extracellular domain selected from the group consisting of residues 33-46, 102-110, 192-210, and 263-350 of SEQ ID NO: 2;
an intracellular domain selected from the group consisting of residues 1-12, 67-78, 134-168, and 234-239 of SEQ ID NO: 2; and
a transmembrane domain selected from the group consisting of residues 13-32, 47-66, 79-101, 111-133,169-191, 211-233, and 240-262 of SEQ ID NO: 2.

3. An isolated CNGH0011 polypeptide, comprising 1-50 amino acid substitutions of SEQ ID NO: 2.

4. An isolated CNGH0011 polypeptide comprising a first portion of the amino acid sequence of SEQ ID NO: 2 wherein a second portion of the amino acid sequence of SEQ ID NO: 2 is deleted.

5. An antagonist to a CNGH0011 polypeptide.

6. The antagonist of claim 5 comprising an antagonist to (i) the polypeptide of SEQ ID NO: 2, or (ii) a fragment of the polypeptide of SEQ ID NO:2.

7. The antagonist of claim 5, comprising at least one member selected from the group consisting of a monoclonal antibody, a polyclonal antibody, a fusion protein, a fragment of an antibody or fusion protein, an siRNA molecule, an shRNA molecule, a DNAzyme molecule, a ribozyme molecule, an aptamer molecule, and an antisense molecule.

8. The antagonist of claim 7, comprising an siRNA molecule selected from the group consisting of SEQ ID NOS: 7-10.

9. The antagonist of claim 5, comprising an anti-CNGH0011 antibody.

10. An isolated nucleic acid molecule encoding the anti-CNGH0011 antibody of claim 9.

11. A vector comprising the isolated nucleic acid molecule of claim 10.

12. A host cell comprising the isolated nucleic acid molecule of claim 10.

13. A method for producing an anti-CNGH0011 antibody, comprising translating the nucleic acid molecule of claim 10, under conditions in vitro, in vivo or in situ, wherein the anti-CNGH0011 antibody is expressed in detectable or recoverable amounts.

14. An antibody produced by the method of claim 13.

15. A composition for diagnosing or treating a CNGH0011-related disorder comprising at least one agonist or antagonist of a protein having the amino acid sequence of SEQ ID NO:2 or a polynucleotide having the nucleotide sequence of SEQ ID NO: 1 selected from the group consisting of a nucleic acid molecule, a polypeptide, and the antibody of claim 9.

16. The composition of claim 15, wherein the agonist or antagonist is selected from the group consisting of the nucleic acid molecule of SEQ ID NO: 1, the polypeptide of SEQ ID NO:2, and an antibody binding to the polypeptide of SEQ ID NO:2.

17. The composition of claim 16, wherein said composition further comprises at least one pharmaceutically acceptable carrier or diluent.

18. The composition of claim 17, administered in combination with at least one composition comprising at least one compound, composition or polypeptide selected from the group consisting of a detectable label or reporter, a TNF antagonist, an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplastic, an immunomodulation drug, an opthalmic, otic or nasal drug, a topical drug, a nutritional supplement, a cytokine, and a cytokine antagonist.

19. The composition of claim 18, in a form of at least one selected from a liquid or gas solution, mixture, suspension, emulsion or colloid, a lyophilized preparation, and a powder.

20. A method for diagnosing a CNGH0011-related condition in a cell, tissue, organ or animal, comprising administering a composition adapted to detect the composition of claim 15.

21. A method for treating a CNGH0011-related condition in a cell, tissue, organ or animal, comprising administering a composition comprising an effective amount of the composition of claim 17, to said cell, tissue, organ or animal.

22. The method of claim 21, wherein the CNGH0011-related condition is a chronic obstructive pulmonary disease-related condition.

23. The method of claim 22, wherein the chronic obstructive pulmonary disease-related condition is asthma.

24. A method for treating a chronic obstructive pulmonary disease-related condition in a cell, tissue, organ or animal, comprising administering a composition comprising an effective amount of at least one member selected from the group consisting of a modulator of CNGH0011 nucleic acid molecule levels, a modulator of CNGH0011 polypeptide levels, and a modulator of CNGH0011 activity, to said cell, tissue, organ or animal.

25. The method of claim 24, wherein said effective amount is about 0.001-50 mg of anti-CNGH0011 antibody; about 0.000001-500 mg of CNGH0011 polypeptide; or about 0.0001-100 μg of CNGH0011 nucleic acid molecule per kilogram of said cells, tissue, organ or animal.

26. The method of claim 25, wherein said administration is by at least one mode selected from the group consisting of parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, and transdermal.

27. The method of claim 26, further comprising administering, prior, concurrently or after said administering step, an effective amount of at least one compound or polypeptide selected from the group consisting of a detectable label or reporter, a TNF antagonist, an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplastic, an immunomodulation drug, an opthalmic, otic or nasal drug, a topical drug, a nutritional supplement, a cytokine, and a cytokine antagonist.

28. A device, comprising the composition of claim 15, wherein said device is suitable for administering the composition, by at least one mode selected from the group consisting of parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, and transdermal.

29. An article of manufacture for human pharmaceutical or diagnostic use, comprising packaging material and a container comprising the composition of claim 15.

30. The article of manufacture of claim 29, wherein said container is a component of a parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal delivery device or system.

31. A method for producing the composition of claim 15, comprising providing at least one member from the group consisting of a vector, host cell, transgenic animal, transgenic plant, and plant cell capable of transcribing said nucleic acid or expressing in detectable or recoverable amounts said polypeptide or antibody.

32. A composition produced by the method of claim 31.

33. A fusion polypeptide comprising the polypeptide of SEQ ID NO:2 or a fragment of the polypeptide of SEQ ID NO:2 operably linked to a heterologous polypeptide.

34. The fusion polypeptide of claim 33 wherein the heterologous polypeptide is selected from a member of the immunoglobulin protein family.

35. A method for detecting the presence or absence of the nucleic acid molecule of SEQ ID NO: 1 or the polypeptide of SEQ ID NO:2 in a biological sample, comprising obtaining a biological sample from a test subject and contacting the biological sample with a compound or an agent capable of detecting said nucleic acid molecule or polypeptide such that the presence of said polypeptide or nucleic acid molecule is detected in the biological sample.

36. A method for antagonizing a CNGH0011 nucleic acid molecule or CNGH0011 polypeptide comprising contacting said CNGH0011 nucleic acid molecule or CNGH0011 polypeptide with at least one member selected from the group consisting of an anti-CNGH011 monoclonal antibody, an anti-CNGH011 polyclonal antibody, a fusion protein, a fragment of the antibody or fusion protein, an siRNA molecule, an shRNA molecule, a DNAzyme molecule, a ribozyme molecule, an aptamer molecule, and an antisense molecule.

37. The method of claim 36, wherein the siRNA molecule is selected from the group consisting of SEQ ID NOS: 7-10.

38. The method of claim 36, wherein said CNGH0011 nucleic acid molecule is the nucleic acid molecule of SEQ ID NO: 1 and said CNGH0011 polypeptide is the polypeptide of SEQ ID NO:2.
